(19) United States
(12) Patent Application Publication
(10) Pub. No.: US 2005/0119197 A1
(43) Pub. Date: Jun. 2, 2005

(54) Naadp analogues for modulating t-cell activity

(76) Inventors: Barry V. L. Potter, Bath (GB); Andreas H. Guse, Hamburg (DE); Georg W. Mayr, Pinneberg (DE); Ingeborg Berg, Ahrensburg (DE)

Correspondence Address: St Onge Steward Johnston & Reens, 986 Bedford Street, Stamford, CT, 06905-5619, US

(21) Appl. No.: 10/343,667

(22) PCT Filed: Jul. 31, 2001
(86) PCT No.: PCT/GB01/03440

(30) Foreign Application Priority Data
Aug. 4, 2000 (GB) 0019234.4

(51) Int. Cl. A61K 31/70; A01N 43/04

(52) U.S. Cl. 514/43

(57) ABSTRACT

A method for modulating T cell activity by modulating the intracellular concentration and/or activity of NAADP+, compounds capable of modulating the effect of NAADP+ on T cell Ca2+ levels, and methods for identifying the same, are described.

### FIELD OF THE INVENTION

The present invention relates to therapeutics. In particular, the present invention relates to the modulation of T cell activity via a nicotinic acid adenine dinucleotide phosphate (NAADP+) mediated pathway. The invention also relates to compounds capable of modulating the activity of T cells via such a pathway. The invention also relates to treating diseases using such compounds and methods for identifying such compounds.

### BACKGROUND TO THE INVENTION

Adaptive or specific immune responses are normally stimulated when an individual is exposed to a foreign antigen. Specific immunity is mediated by lymphocytes, e.g. B and T lymphocytes. During an immune response, recognition of an antigen leads to activation of lymphocytes that specifically recognise that particular antigen. The lymphocytes proliferate and differentiate into specialised effector cells. The immune response culminates in the development of mechanisms that ultimately eliminate the antigen.

Adaptive immune responses are critical components of host defence during protection against foreign antigens, such as infectious organisms or toxins. However, specific immune responses are also sometimes elicited by antigens not associated with infectious agents, and this may cause serious disease. For example, one of the most remarkable properties of specific immunity is the ability to distinguish between self antigens and foreign antigens. Thus, the lymphocytes in each individual are able to recognise and respond to numerous foreign antigens but are normally unresponsive to potentially antigenic substances present in the individual itself Unresponsiveness to self antigens is an acquired process that has to be learned by the individual's lymphocytes and has to be maintained throughout life.

Abnormalities in the induction or maintenance of self-tolerance lead to immune responses against self antigens, and debilitating diseases that are commonly called autoimmune diseases. The spectrum of autoimmune disorders ranges from organ specific diseases (such as thyroiditis, insulitis, multiple sclerosis, iridocyclitis, uveitis, orchitis, hepatitis, Addison's disease, myasthenia gravis) to systemic illnesses such as rheumatoid arthritis or lupus erythematosus.

Another example in which specific immunity against antigens that are not associated with infections causes severe medical problems are rejections of transplanted allografts. In fact, adaptive immune responses to grafted tissues are the major impediment to successful transplantation in most cases.

It is not known what causes the breakdown of tolerance and the initiation of an autoimmune response. However, the mechanisms of tissue destruction in autoimmune diseases and in allograft rejection are essentially the same as those operating in protective immunity. It is generally believed that both autoimmune reactions and allograft rejections are initiated and perpetuated by a response involving T cells. Thus, in the absence of a specific therapy for any of the autoimmune diseases or for allograft rejection, many therapeutic strategies currently employed aim at down modulating the activity of the immune system, in particular by reducing or preventing the activation of T cells.

Recently, monoclonal antibodies to T cell surface antigens, that inhibit T cell activation, or substances that interfere with intracellular T cell activation pathways, such as Cyclosporin A or FK506, have been introduced for the treatment of both allograft rejection and several autoimmune diseases. However, current approaches for the treatment of undesirable T cell activation have been associated with a number of side effects related to general immunosuppression and therefore cannot be considered to be optimal therapy.

Stimulation of T-lymphocytes via the T cell receptor/CD3 complex (TCR/CD3) is a critical step in T cell activation and subsequent clonal expansion. Previous studies have shown that activation of the TCR/CD3-complex involves the elevation of the free cytosolic Ca²⁺ concentration ([Ca²⁺]ᵢ) by at least two mechanisms, a rapid elevation caused by Ca²⁺ release from intracellular stores mediated by inositol (1,4,5) trisphosphate (Ins(1,4,5)P₃), and a prolonged elevation that is completely dependent on the influx of extracellular calcium (reviewed in Guse, 1998). Ca²⁺-release is activated by the calcium mobilizing second messengers Ins(1,4,5)P₃ (Jayaraman et al, 1995) and cADPR (Guse et al., 1999). Recent work indicates that Ins(1,4,5)P₃ primarily acts during the initial phase of Ca²⁺-signaling in T cells, whereas cADPR is essentially involved in the sustained phase of Ca²⁺-signaling.

The exact mechanism of Ca²⁺ signalling in T cells is still unclear, but it is of fundamental importance for proliferation and clonal expansion, and thus for a functional immune response. An improved understanding of the signalling pathways involved in T cell activation may be of assistance in developing strategies to stimulate a desirable adaptive immune response or to suppress inappropriate T cell activity.

### SUMMARY OF THE INVENTION

The present inventors have shown that NAADP+ specifically and dose-dependently stimulates Ca²⁺ signalling in human T cells. At an activating concentration, NAADP+ either evokes repetitive and longlasting Ca²⁺ oscillations or a single Ca²⁺-spike of high amplitude.

The present inventors have also shown that NAADP+ can be self-inactivating. An inactivating concentration of NAADP+ inhibits subsequent stimulation of Ca²⁺ signaling via the T cell receptor/CD3. For example, inactivation of the NAADP⁺/Ca²⁺-release system almost completely abolishes subsequent Ins(1,4,5)P₃- or cADPR-mediated Ca²⁺-signaling. This shows that a functional NAADP+/Ca²⁺ release system is essential for T-lymphocyte Ca²⁺ signaling.

These findings have important implications for the design of compounds capable of modulating T cell activity, since regulation of this NAADP+/Ca²⁺ signalling pathway may provide an important means of stimulating T cells (and adaptive immune responses) and controlling T cell responses in a variety of T cell mediated immune disorders.

Accordingly the present invention provides a method for modulating T cell activity, which comprises the step of modulating the intracellular concentration of NAADP+ or a bioisostere thereof.

In one embodiment, the method involves stimulating a rise in intracellular Ca²⁺ levels by raising the intracellular concentration of NAADP+ to an activating concentration. The present inventors have found that an intracellular concentration of 10 nM NAADP+ evokes repetitive and longlasting Ca²⁺ oscillations of low amplitude, while 50 and 100 nM produces a rapid and high initial Ca²⁺ peak followed by trains of smaller Ca²⁺ oscillations. Higher concentrations of NAADP+(1 and 10 μM) gradually reduce the initial Ca²⁺ peak. Thus an “activating concentration” of NAADP+ may be between 5 nM and 1 μM, preferably between 5 and 100 nM.

In another embodiment, the method involves inhibiting TCR/CD3-associated Ca²⁺ signaling by raising the intracellular concentration of NAADP+ to an inactivating concentration. The present inventors have shown that an intracellular concentration of 100 μM NAADP+ causes complete self-inactivation of Ca²⁺-signals. Thus an “inactivating concentration” of NAADP+ may be greater than 1 μM, preferably greater than 10 M, most preferably 100 μM or greater.

The elucidation of a novel NAADP+-mediated T cell activation pathway also enables the identification of substances that modulate T cell activation via this pathway.

The present invention thus also provides a compound capable of
(a) antagonising the NAADP+-mediated rise in intracellular Ca²⁺ levels caused by TCR/CD3 stimulation;
(b) agonising the NAADP+-mediated rise in intracellular Ca²⁺ levels caused by TCR/CD3 stimulation;
(c) inducing the NAADP+-mediated inhibition of TCR/CD3-associated Ca²⁺ signaling; or
(d) preventing the NAADP+-mediated inhibition of TCR/CD3-associated Ca²⁺ signaling

Compounds of the invention which inhibit T cell proliferation and/or differentiation, or induce T cell anergy may be used in treating diseases characterised by an excessive or inappropriate T cell response, such as autoimmune diseases, allergies and allograft rejection. Candidate autoimmune diseases include thyroiditis, insulitis, multiple sclerosis, iridocyclitis, uveitis, orchitis, hepatitis, Addison's disease, myasthenia gravis, rheumatoid arthritis and lupus erythematosus.

Compounds of the invention which induce or enhance T cell proliferation and/or differentiation or prevent the induction of T cell anergy may be used generally to boost or induce T cell immune responses. Virtually all adaptive immune responses require the activation of T cells and their differentiation into cytokine-producing cells. Thus these compounds may be used generally to prevent and treat conditions such as infectious diseases (such as viral or bacterial infections), cancers and, in particular, immunodeficiencies characterised by impaired T cell function (such as AIDS).

The present invention further provides a method for identifying a substance capable of antagonising the NAADP+-mediated rise in intracellular Ca²⁺ levels in a T cell, which method comprises:
(i) contacting a T cell, which has been stimulated via its T cell receptor, with a candidate substance under conditions that would permit a sustained rise in intracellular Ca²⁺ levels in the absence of the substance; and
(ii) determining whether the substance inhibits a sustained rise in intracellular Ca²⁺ levels.

In one embodiment, the substance inhibits NAADP+ synthesis, for example reduces or abolishes NAADP+ synthesis. In another embodiment, the substance modulates, for example inhibits, binding of endogenous NAADP+ to its receptor binding site.

The present invention further provides a method for identifying a substance capable of inducing the NAADP+-mediated inhibition of TCR/CD3-associated Ca²⁺ signalling, which method comprises:
(i) contacting a T cell with a candidate substance;
(ii) stimulating the T cells via TCR/CD3; and
(ii) determining whether the substance inhibits TCR/CD3-associated Ca²⁺ signalling.

In one embodiment, the substance causes the intracellular concentration of NAADP+ (or a bioisostere thereof) to rise to an inactivating concentration.

The present invention further provides a method for identifying a substance capable of agonising the NAADP+-mediated rise in intracellular Ca²⁺ levels in a T cell, which method comprises:
(i) contacting a T cell with a candidate substance; and
(ii) determining whether the substance elicits or enhances a rise in intracellular NAADP+ and/or Ca²⁺ levels.

In one embodiment, the substance induces or enhances NAADP+ synthesis. In another embodiment, the substance modulates, for example enhances, binding of endogenous NAADP+ to its receptor binding site.

A compound identified by the methods of the invention may be used in modulating the immune response of a mammal. Thus, for example, in another aspect of the present invention, a compound identified by a method of the invention is provided for use in treating (i) an autoimmune disease, such as thyroiditis, insulitis, multiple sclerosis, iridocyclitis, uveitis, orchitis, hepatitis, Addison's disease, myasthenia gravis, rhematoid arthritis and lupus erythematosus or (ii) allograft rejection.

The present invention also provides a pharmaceutical composition (which term also includes a veterinary formulation) comprising a compound of the present invention, or a pharmaceutically acceptable salt thereof, or a pharmaceutically acceptable solvate of either entity, together with a pharmaceutically acceptable diluent, excipient or carrier.

The invention further provides a compound of the present invention, or a pharmaceutically acceptable salt thereof, or a pharmaceutically acceptable solvate of either entity, or a pharmaceutical composition containing any of the foregoing, for use as a human or animal medicament.

### DETAILED DESCRIPTION OF THE INVENTION

A. Modulation of the Intracellular Concentration of NAADP+ or a Bioisostere Thereof

NAADP⁺ was originally discovered as a contaminant of commercial NADP⁺ preparations; such preparations could also be enriched in NAADP⁺ content by alkaline treatment (Clapper et al., 1987). NAADP⁺ has the following formula:

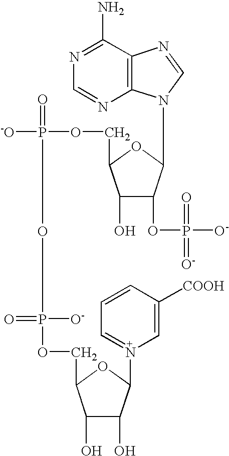

NAADP+ has been shown to be involved in Ca²⁺-mobilisation in some invertebrate cell types, such as sea urchin eggs (Lee & Aarhus 1995), ascidian oocytes (Albrieux et al., 1998), and more recently in mouse pancreatic acinar cells (Cancela et al., 1999). To date, NAADP+ has not been shown to have an effect in a human cell system.

The actual mechanism of formation of NAADP+ in vivo is unknown, but at least three enzyme activities have been implicated: firstly, a deamidase enzyme which cleaves the amide group of (nicotinamide-adenine dinucleotide phosphate) NADP; and secondly a kinase enzyme that phosphorylates the 2′-position of nicotinic acid-adenine dinucleotide (NAAD). It is also thought that ADP-ribosyl cyclase may play a role, since it is known to be able to make NAADP+ in vitro from NADP+ by base exchange.

The pathway by which NAADP+ may be eliminated from the cell is unclear. It may involve 2′-dephosphorylation, cleavage at the pyrophosphate group and/or loss of the nicotinic acid group to give ADPRP.

The present inventors have shown that NAADP+ is capable of
(a) stimulating Ca²⁺ signalling in human T cells, when present at an activating concentration; and
(b) self-inactivating the NAADP⁺/Ca²⁺-release system when present at an inactivating concentration.

As used herein, the term “bioisostere” is used to indicate a compound which is structurally distinct from NAADP+, but which shares some functional similarity with NAADP+. The bioisostere may be capable of performing one or more of the biological functions of NAADP+. For example, the bioisostere may be capable of stimulating Ca²⁺ signalling in human T cells via the NAADP⁺/Ca²⁺-release system. Alternatively, or in addition, the bioisostere may be capable of inhibiting the NAADP⁺/Ca²⁺-release system, for example, by mimicking the effect of NAADP+ when present at an inactivating concentration.

A bioisostere may be endogenous to the T cell, or may be artificially introduced. A bioisostere may be any compound capable of exhibiting the required functional characteristics. For example, the bioisostere may be a small organic molecule, possibly sharing some structural features with NAADP⁺. Preferable the bioisostere shows significant structural similarity to NAADP+.

There are a number of methods by which the intracellular concentration of NAADP+ (or a bioisostere thereof) may be modulated.

The concentration may be increased directly by administering NAADP+ or a bioisostere thereof or a precursor thereof to the cell. For example, the compound may be administered by any of the known delivery routes, such as microinjection (as described in the Examples), delivery via a carrier protein, or liposome-mediated delivery.

Alternatively, the intracellular concentration may be increased by activating one or more steps in the intracellular NAADP+ production pathway, or by inhibiting NAADP+ breakdown and/or escape from the cell. For example, NAADP+ levels may be raised by activating one or more of the enzymes involved in NAADP+ synthesis (such as the above-mentioned deamidase enzyme, kinase enzyme and/or ADP-ribosyl cyclase).

Conversely, the concentration of NAADP+ may be decreased by blocking one or more steps in the intracellular NAADP+ production pathway, or by activating NAADP+ breakdown and/or escape from the cell. For example, NAADP+ production may be blocked by inhibiting one or more of the enzymes involved in NAADP+ synthesis (such as the deamidase enzyme, kinase enzyme and/or ADP-ribosyl cyclase).

Also, the effective concentration of NAADP+ may be decreased by blocking the interaction between NAADP+ and its receptor. For example, the presence of a substance which binds to NAADP+, inhibiting or preventing its interaction with its receptor will decrease the effective concentration of NAADP+ in a cell.

In one embodiment, the method of the present invention involves stimulating a rise in intracellular Ca²⁺ levels by raising the intracellular concentration of NAADP+, or a bioisostere thereof, to an activating concentration. An “activating concentration” of NAADP+ is an intracellular concentration of between 5 nM and 1 μM, preferably between 5 and 100 nM, more preferably about 10 nM.

An “activating concentration” of a bioisostere is that concentration which causes a comparable rise in intracellular Ca²⁺ levels to that caused by an activating concentration of NAADP+. The actual concentration will depend on the chemical nature of the bioisostere. It may be comparable to the “activating concentration” of NAADP+, especially if the bioisostere is of a similar chemical structure.

In another embodiment, the method involves inhibiting TCR/CD3-associated Ca²⁺ signaling by raising the intracellular concentration of NAADP+ to an inactivating concentration. An “inactivating concentration” of NAADP+ is an intracellular concentration of greater than 1 μM, preferably greater than 10 μM, most preferably 100 μM or greater.

An “inactivating concentration” of a bioisostere is that concentration which inhibits TCR/CD3-associated Ca²⁺ signaling by blocking the NAADP+/Ca²⁺ release system to a degree which is comparable to that caused by an inactivating concentration of NAADP+. The actual concentration will depend on the chemical nature of the bioisostere. It may be comparable to the “inactivating concentration” of NAADP+, especially if the bioisostere is of a similar chemical structure.

B. Compounds Capable of Modulating NAADP+ Mediated Ca²⁺ Signalling in T Cells.

Compounds suitable for use in the present invention are capable of modulating NAADP+-mediated Ca²⁺ signalling in T cells. They may act as antagonists, by inhibiting or blocking the NAADP+-mediated rise in intracellular Ca²⁺ levels which occurs in response to TCR/CD3 stimulation on the T cell surface.

An antagonist may cause the intracellular concentration of NAADP+ to fall, or prevent the intracellular concentration to rise to an activating concentration. Alternatively, the antagonist may act on other components of the NAADP+ mediated Ca²⁺ release pathway.

Preferably, an antagonist for use in the present invention should not substantially inhibit other pathways involved in the release of Ca²⁺ from intracellular stores (for example Ins(1,4,5)P₃ mediated release) or involved in the influx of Ca²⁺ (for example cADPR-mediated influx). For example, a preferred antagonist will inhibit the NAADP+ pathway at least two-fold, preferably at least 5 or 10-fold more than other pathways involved in Ca²⁺ signalling resulting from TCR/CD3 stimulation.

A compound for use in the present invention may act at a number of places in the NAADP+ Ca²⁺ signalling pathway. It may affect signalling between the activated TCR/CD3 complex and an NAADP+-producing enzyme (for example the above-mentioned deamidase enzyme, kinase enzyme or ADP-ribosyl cyclase). It may affect activation of the NAADP+-producing enzyme (such as covalent modification by an upstream effector protein, for example a kinase), or enzymatic activity of the NAADP+-metabolising enzyme (such as a competitive inhibitor having a high binding affinity for the active site of the enzyme or a non-competitive inhibitor which binds a distal site resulting in a conformational change). Alternative, it may affect downstream effects of NAADP+. In particular preferred compounds such as NAADP+ analogues may act to inhibit binding of NAADP+ to its binding site on its receptor. Suitable assays for identifying compounds for use in the present invention are described below in section D.

One particularly preferred class of compounds for use in the present invention are NAADP+ analogues, which may bind to the receptor and compete with NAADP+.

Compounds suitable for use in the present invention may alternatively act as agonists, by stimulating or enhancing the NAADP+-mediated rise in intracellular Ca²⁺ levels which occurs in response to TCR/CD3 stimulation on the T cell surface. An agonist may act by raising intracellular concentrations of NAADP+ to an activating concentration or by affecting other components in the NAADP+ Ca²⁺ signaling pathway (as explained above with regard to antagonists).

A compound may also be capable of inducing or blocking the NAADP+-mediated inhibition of TCR/CD3-associated signalling. For example, the compound may be capable of raising, or blocking the elevation of, intracellular NAADP+ concentrations to an inactivating concentration. Alternatively, such a compound may act at another step in the auto-inactivation pathway.

Compounds suitable for use in the present invention include NAADP analogues having the following formula (I): 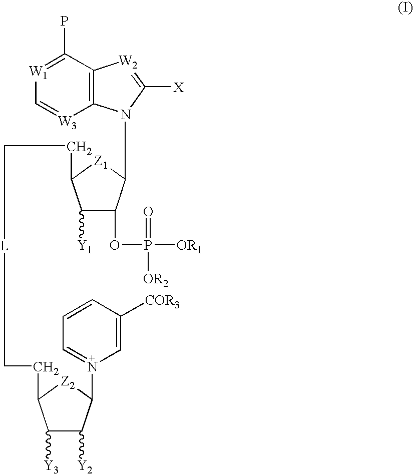
wherein P is a substituent group independently selected from NH₂, OH, SH; each of W1, W2 or W3 is independently selected from either a CH or a heteroatom, such as N, P, S or O, preferably N;
X is a substituent group independently selected from OH, SH, NH₂, or a halo group (preferably Br);
each of R₁, R₂ or R₃ is independently selected from H or 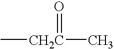
each of Y₁, Y₂ or Y₃ is independently selected from OH, H, NH₂, halo (preferably F), SH, OR₄, or 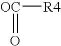
wherein R₄ is a hydrocarbyl; each of Z₁ or Z₂ is independently selected from O, S, CH₂ or a halo derivative thereof, preferably CF₂;
and L is a linker group, suitably the linker group may have the formula (II): 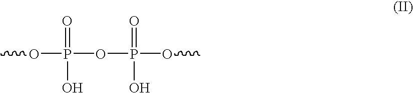
   or may be selected from one ore more the group comprising: a phosphate, a polyphosphate, a phosphorothioate, a polyethylene glycol, an alkyl, an alkylaryl, a peptide and a polyamine;
   or isomeric forms of the compound of Formula (I).

In a preferred aspect of the present invention the compound may 8-bromo-nicotinic acid adenine dinucleotide phosphate (8-Br-NAADP). 8-Br-NAADP modulates the intracellular concentration and/or the binding affinity of NAADP+.

The term “compound” is intended to encompass isomeric forms (such as stereoisomers and/or geometric and/or optical isomers, and mixtures thereof), chemical derivatives, mimetics, solvates and salts of the compounds.

As used herein, the term “hydrocarbyl” refers to a group comprising at least C and H that may optionally comprise one or more other suitable substituents. Examples of such substituents may include halo-, alkoxy-, nitro-, an alkyl group, or a cyclic group. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain heteroatoms. Suitable heteroatoms will be apparent to those skilled in the art and include, for instance, sulphur, nitrogen, oxygen, silicon and phosphorus.

For some embodiments, preferably the hydrocarbyl group is alkyl, alkoxy, alkenyl, alkylene, acyl and alkenylene groups—which may be unbranched- or branched-chain.

For some embodiments, preferably the hydrocarbyl group is C₁₋₁₂ alkyl, C₁₋₁₂ alkoxy, C₁₋₁₂ alkenyl, C₁₋₁₂ alkylene, C₁₋₁₂ acyl, and C₁₋₁₂ alkenylene groups—which may be unbranched- or branched-chain.

For some embodiments, preferably the hydrocarbyl group is C₁₋₆ alkyl, C₁₋₆ alkoxy, C₁₋₆ alkenyl, C₁₋₆ alkylene, C₁₋₆ acyl, and C₁₋₆ alkenylene groups—which may be unbranched- or branched-chain.

It is to be appreciated that all references herein to treatment include one or more of curative, palliative and prophylactic treatment. Preferably, the term treatment includes at least curative treatment and/or palliative treatment.

C. Chemical Synthesis Methods.

The compounds of the present invention may be available commercially.

Alternatively, the compound of the invention may be prepared by chemical synthesis techniques.

It will be apparent to those skilled in the art that sensitive functional groups may need to be protected and deprotected during synthesis of a compound of the invention. This may be achieved by conventional techniques, for example as described in “Protective Groups in Organic Synthesis” by T W Greene and P G M Wuts, John Wiley and Sons Inc. (1991), and by P. J. Kocienski, in “Protecting Groups”, Georg Thieme Verlag (1994).

It is possible during some of the reactions that any stereocentres present could, under certain conditions, be epimerised, for example if a base is used in a reaction with a substrate having an having an optical centre comprising a base-sensitive group. It should be possible to circumvent potential problems such as this by choice of reaction sequence, conditions, reagents, protection/deprotection regimes, etc. as is well-known in the art.

The compounds and salts of the invention may be separated and purified by conventional methods.

D. Synthesis of 8-bromo-nicotinic Acid Adenine Dinucleotide Phosphate (8-Br-NAADP).

Synthesis of 8-Br-NADP

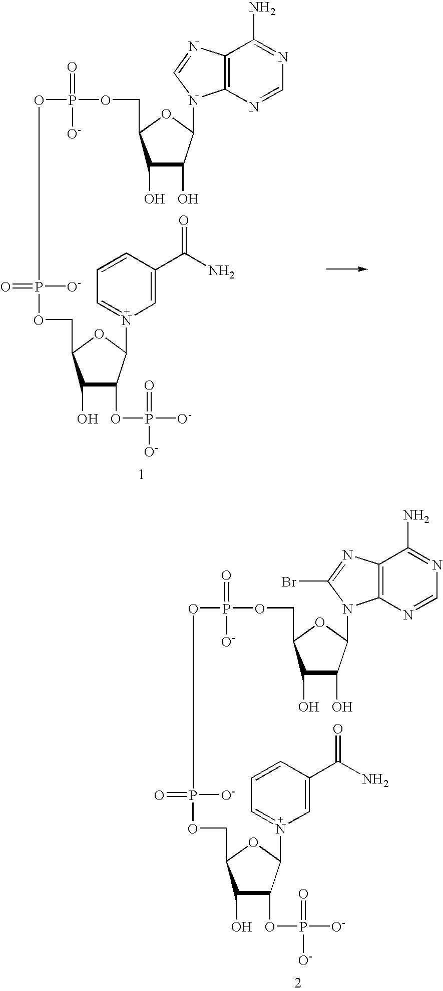

To a solution of nicotinamide adenine dinucleotide phosphate sodium salt 1 (50 mg, 0.065 mmol) in 1M aqueous sodium acetate pH 4 buffer (2 ml) bromine (50 μl, 0.975 mmol) was added via syringe (Holmes et al. J. Am. Chem. Soc., 86, 1242, (1964)). The reaction mixture was stirred in the dark at ambient temperature for two hours, after which time high performance liquid chromatography (HPLC) analysis (Rainin Dynamix SD-200 HPLC system using a Supelcosil™ Saxi ion exchange column (25 cm×4.6 mm, 5 μm), and eluting with 50 mM aqueous potassium hydrogen phosphate pH 3 buffer with 5% methanol) indicated that all the nicotinamide adenine dinucleotide phosphate sodium salt 1 (R_{T}=11 mins) had been consumed. The excess bromine was extracted into chloroform (3×5 ml), and the aqueous phase frozen and lyophilised to produce the crude 8-Br-nicotinamide adenine dinucleotide phosphate sodium salt 2 (R_{T}=12 mins) as a pale yellow powder in 98% yield by HPLC analysis (see above). The crude product was purified by semi-preparative HPLC (Rainin Dynamix SD-200 HPLC system using a Supelcosil™ LC-Saxi ion exchange column (25 cm×10 mm, 5 μm), and eluting with 50 mM aqueous potassium hydrogen phosphate pH 3 buffer with 5% methanol). Inorganic phosphate was removed from the product by LiChroprep® reverse phase (25-40 μm) column chromatography (25 mM aqueous triethylammonium formate solution), and monitored by the use of an Aquamerck® (14661) blue colour test (Hoffmann et al. Bioorg. & Med. Chem. Lett., 6, 2571, (1996)). The desired product (namely 8-Br-NADP 2) eluted with Milli-Q water and was isolated as its triethylammonium salt; δ_{H} 400 MHz (D₂O) 4.05 (2H, m, 2H_{A}5′), 4.14 (3H, m, 2H_{N}5′, H_{A}4′), 4.26 (4H, m, H_{N}4′, H_{A}3′, H_{N}3′, H_{N}2′), 5.25 (1H, d, J 5.0, H_{A}2′), 5.74 (1H, d, J 5.0, H_{A}1′), 5.89 (1H, d, J 5.0, H_{N}1′), 7.94 (1H, s, H_{A}2), 7.99 (1H, m, H_{N}⁵), 8.60 (1H, d, J 8.0, H_{N}⁴), 8.88 (1H, d, J6.0, H_{N}6), 9.12 (1H, s, H_{N}2).
Synthesis of 8-Br-NAADP 3 (Route 1)

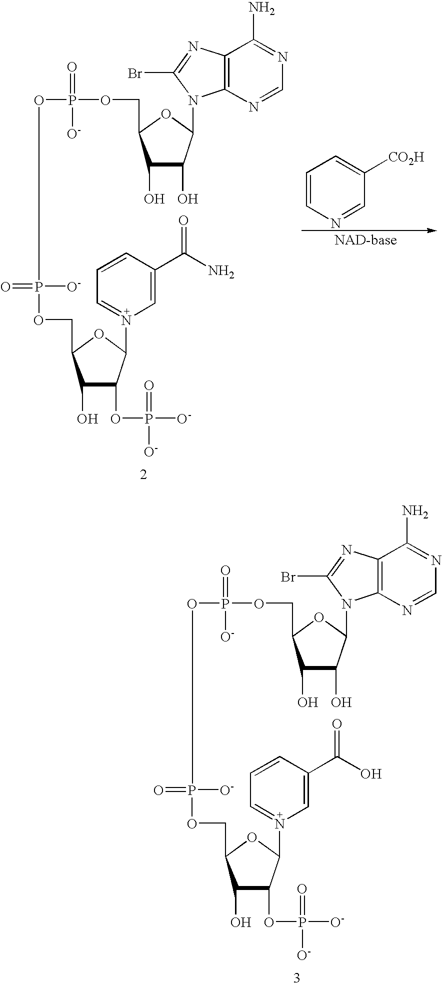

The title compound 3 was synthesised using a modification of the Bernoksky procedure for nicotinic acid dinucleotide phosphate synthesis (Bemofsky et al. Analytical Biochemistry, 67, 611, (1975) and Bemofsky Method. Enzymol., 66, 105, (1980)). NAD-ase (150 mg) was suspended in 50 mM aqueous triethanolamine acetate pH 7.6 buffer (2 ml) and sonicated for 10 mins to induce homogenisation. This solution was then added to 8-bromo-nicotinamide adenine dinucleotide phosphate triethylammonium salt 2 (50 mg, 0.059 mmol) and nicotinic acid (250 mg, 2.03 mmol). The reaction was stirred at 37° C. for 14 hrs. HPLC analysis (see above) showed consumption of 8-Br-nicotinamide adenine dinucleotide phosphate triethylammonium salt 2 (R_{T}=12 mins) and formation of 8-bromo-nicotinic acid adenine dinucleotide phosphate 3 (R_{T}=25 mins). The crude reaction mixture was filtered through celite and purified by semi-preparative HPLC (see above). Inorganic phosphate was removed from the product by LiChroprep® reverse phase (25-40 μm) column chromatography (25 mM aqueous triethylammonium formate solution), and monitored by the use of an Aquamerck® (14661) blue colour test (see above). The desired product (namely 8-Br-NAADP 3) eluted with Milli-Q water and was isolated as its triethylammonium salt; δ_{H} 400 MHz (D₂O) 4.10 (3H, m, 2H_{N}5′, H5_{A}′), 4.25 (1H, m, H5_{A}′), 4.32 (1H, m, H_{A}⁴′), 4.39 (1H, m, H_{N}4′), 4.45 (1H, m, H_{N}3′), 4.51 (1H, m, H_{N}2′), 4.60 (1H, m, H_{A}3′), 5.01 (1H, m, H_{A}2′), 5.97 (1H, br s, H_{N}′), 6.06 (1H, d, J6.0, H_{A}1′), 8.12 (1H, m, H_{N}⁵), 8.26 (1H, s, H_{A}2), 8.85 (1H, d, J7.5, H_{N}4), 9.09 (1H, d, J5.0, H_{N}6), 9.28 (1H, s, H_{N}2).

Synthesis of 8-Br-NAADP 3 via Direct Bromination (Route 2)

i. Preparation of NAADP 4

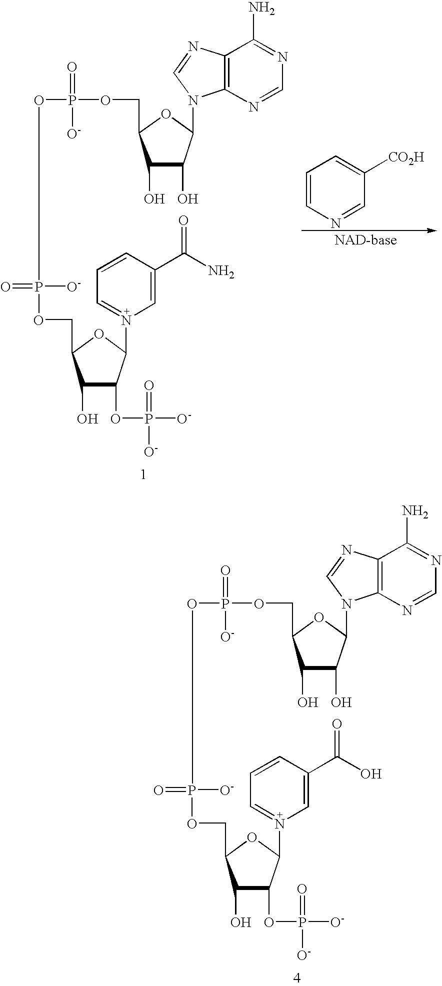

Nicotinic acid adenine dinucleotide phosphate 4 was synthesised using a modification of the Bernoksky procedure (see above). NAD-ase (90 mg) was suspended in 50 mM aqueous triethanolamine acetate pH 7.6 buffer (2 ml) and sonicated for 10 mins to induce homogenisation. This solution was then added to nicotinamide adenine dinucleotide phosphate sodium salt 1 (30 mg, 0.039 mmol) and nicotinic acid (240 mg, 1.96 mmol). The reaction was stirred at 37° C. for 14 hrs. HPLC analysis (see above) showed consumption of nicotinamide adenine dinucleotide phosphate sodium salt 1 (R_{T}=12 mins) and formation of nicotinic acid adenine dinucleotide phosphate 4 (R_{T}=17 mins). The crude reaction mixture was filtered through celite and purified by AG-MP1 ion-exchange resin (150 mM aqueous TFA solution gradient). The desired product (namely NAADP 4) eluted with 100% 150 mM TFA solution; δ_{H} 400 MHz (D₂O) 4.13 (3H, m, 2H_{N}5′, H5_{A}′), 4.26 (1H, m, H5_{A}′), 4.30 (1H, m, H_{A}⁴′), 4.34 (1H, m, H_{N}4′), 4.44 (1H, m, H_{N}3′), 4.47 (1H, m, H_{N}2′), 4.54 (1H, m, H_{A}3′), 5.01 (1H, m, H_{A}2′), 6.06 (1H, d, J 5.5, H_{N}1′), 6.17 (1H, d, J5.5, H_{A}1′), 8.18 (1H, m, H_{N}5), 8.31 (1H, s, H_{A}2), 8.48 (1H, s, H_{A}8), 8.91 (1H, d, J 8.0, H_{N}4), 9.21 (1H, d, J6.0, H_{N}6), 9.35 (1H, s, H_{N}2).
ii. Bromination of NAADP 4

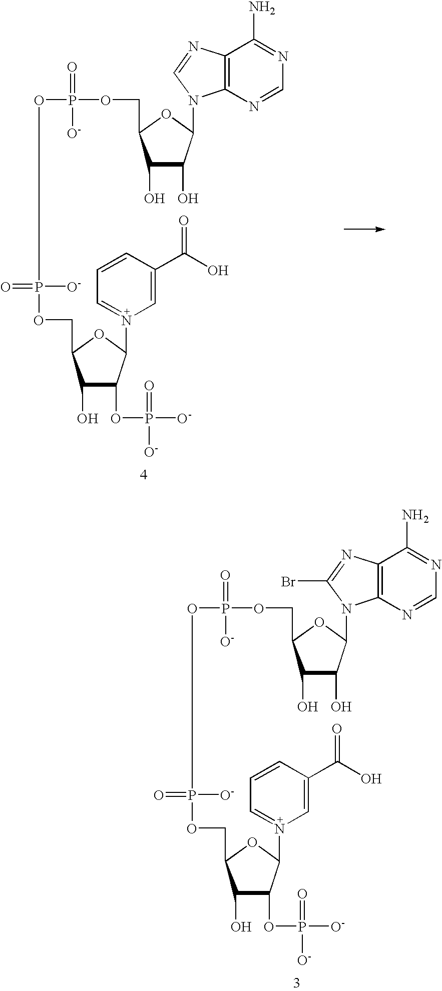

To a solution of nicotinic acid adenine dinucleotide phosphate sodium salt 4 (20 mg, 0.026 mmol) in 1M aqueous sodium acetate pH 4 buffer (2 ml) was added bromine (20 μl, 0.388 mmol) via syringe (see Holmes detailed above). The reaction mixture was stirred in the dark at ambient temperature for two hours, after which time HPLC analysis (see above) indicated that all the nicotinic acid adenine dinucleotide phosphate sodium salt 4 (R_{T}=17 mins) had been consumed. The excess bromine was extracted into chloroform (3×5 ml), and the aqueous phase frozen and lyophilised to produce the crude 8-Br-nicotinic acid adenine dinucleotide phosphate sodium salt 3 R_{T}=25 mins) as a pale yellow powder. The crude product was purified by semi-preparative HPLC (see above). Inorganic phosphate was removed from the product by LiChroprep® reverse phase (25-40 μm) column chromatography (25 mM aqueous triethylammonium formate solution), and monitored by the use of an Aquamerck® (14661) blue colour test (see above). The desired product (namely 8-Br-NAADP 3) eluted with Milli-Q water and was isolated as its triethylammonium salt; δ_{H} 400 MHz (D₂O) 4.10 (3H, m, 2H_{N}5′, H5_{A}′), 4.25 (1H, m, H5_{A′),} 4.32 (1H, m, H_{A}4′), 4.39 (1H, m, H_{N}4′), 4.45 (1H, m, H_{N}3′), 4.51 (1H, m, H_{N}2′), 4.60 (1H, m, H_{A}3′), 5.01 (1H, m, H_{A}2′), 5.97 (1H, br s, H_{N}1′), 6.06 (1H, d, J 6.0, H_{A}1′), 8.12 (1H, m, H_{N}5), 8.26 (1H, s, H_{A}2), 8.85 (1H, d, J 7.5, H_{N}4), 9.09 (1H, d, J 5.0, H_{N}6), 9.28 (1H, s, H_{N}2).

E. Stereo and Geometric Isomers.

Some of the compounds of the present invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those compounds, and mixtures thereof. The terms used in the claims encompass these forms, provided said forms retain the appropriate functional activity (though not necessarily to the same degree).

F. Assays for Identifying Compounds Capable of Modulating NAADP+-Mediated Ca²⁺ Signalling in T Cells.

A substance which affects the NAADP+ pathway may do so in several ways as discussed above.

An antagonist may disrupt an interaction between two components of the pathway, such as disrupting the binding of NAADP+ to its binding site on the receptor. It may directly disrupt the binding of the two components by, for example, binding to one component and masking or altering the site of interaction with the other component. Candidate substances of this type may conveniently be screened by in vitro binding assays. Examples of candidate, substances include non-functional homologues of either of the two components as well as antibodies which recognize either of the two components.

An agonist may enhance the interaction between two components of the pathway. For example, the agonist may bind to one or other component, inducing a conformational change which enhances the interaction between the two components.

A substance which can bind directly to either of the two components may also inhibit or enhance an interaction between the two components by altering their subcellular localization thus preventing or enabling the two components from coming into contact within the cell. This can be tested in vivo using, for example the in vivo assays described below. The term ‘in vivo’ is intended to encompass experiments with cells in culture as well as experiments with intact multicellular organisms.

Alternatively, instead of preventing or enhancing the association of the components directly, the substance may suppress or enhance the biologically available amount of one or both of the components. This may be by inhibiting or enhancing expression of the component, for example at the level of transcription, transcript stability, translation or post-translational stability. An example of such a substance would be antisense RNA which suppresses the amount of mRNA translated into protein of an enzyme involved in NAADP+ synthesis or of the NAADP+ receptor.

Suitable candidate substances also include antibody products (for example, monoclonal and polyclonal antibodies, single chain antibodies, chimeric antibodies and CDR-grafted antibodies) which are specific for either of the two components.

Furthermore, combinatorial libraries, peptide and peptide mimetics, defined chemical entities, oligonucleotides, and natural product libraries may be screened for activity as inhibitors or enhancers of an interaction between the two components. Other candidate substances include analogues of substrates or products of the NAADP+ pathway, such as NAADP+ or NAAD+ analogues.

The candidate substances may be used in an initial screen in batches of, for example 10 substances per reaction, and the substances of those batches which show inhibition tested individually. Candidate substances which show activity in in vitro screens such as those described below may then be tested in in vivo systems.

In vitro assays typically test for substances capable of affecting the interaction between particular components of the signalling pathway (see above) or the activity of a particular component, such as an enzyme. For example, one assay may involve testing candidate substances for the ability to inhibit synthesis of NAADP+. This may be performed by (i) contacting an NAADP+ producing-enzyme with a candidate substance under conditions that would allow the synthesis of NAADP+ in the absence of the candidate substance and (ii) determining if the candidate substance inhibits NAADP+ synthesis.

Other in vitro assays may include assays for identifying substances capable of disrupting NAADP+ binding to its receptor. For example, membrane preparations may be obtained from T cells by methods known in the art and the binding of NAADP+ to the membrane preparation assessed in the absence or presence of a candidate substance.

In vitro assays will generally be used as a preliminary step prior to in vivo testing since it is only in the context of an intact TCR/CD3 Ca²⁺ signalling pathway that the modulatory effects of a candidate substance may be completely assessed. In vivo assays will typically use either T cell lines, preferably human T cell lines, or T cells obtained from animal tissues, especially human tissues. Generally, T cells will be stimulated via the TCR/CD3 complex using standard methods such as receptor cross-linking using antibodies that recognise the receptor complex. For example, the TCR/CD3 complex may be stimulated by the anti-CD3 monoclonal antibody OKT3, as described in Example 4. The TCR/CD3 complex may also be stimulated using superantigens or antigen presenting cells, such as dendritic cells.

A suitable assay method comprises stimulating a T cell via its TCR/CD3 receptor in the presence or absence of a candidate substance. The Ca²⁺ response is measured (for example using ratiometric Ca²⁺ imaging as described in the Examples) and compared. The intracellular concentration of NAADP+ may be measured by labeling of NAADP⁺ by a fluorescent dye (pre- or post-column derivatization) combined with a very sensitive fluorescence detector, e.g. laser-induced fluorescence detection (Rahavendran & Karnes, 1993). Alternatively, a competitive protein binding assay based on a high affinity binding protein for NAADP⁺ may be used.

T cells may be contacted with a candidate substance before, concomitant with, or after stimulation of the TCR/CD3 complex. The concentration of candidate substance administered to the cell will vary but is typically from 0.1 to 100 μM, more preferably from 1 to 100 μM or 10 to 100 μM.

G. Assays for Testing the Physiological Affects of Substances Capable of Modulating NAADP+/Ca²⁺-Release System.

Since the intention of identifying substances that affect the NAADP+ pathway is to use them to modulate T cell activity, further assays may comprise administering a substance capable of modulating the activity of the pathway to a T cell, or a candidate substance, and determining the effect on the cell.

For example, compounds for use in the present invention may be capable of inhibiting or enhancing the NAADP+-mediated rise in Ca²⁺ levels following stimulation of the cell via the TCR/CD3 complex. Such an affect may include inhibition or stimulation of cell proliferation in response to, for example, stimulation by an antigen presenting cell such as a dendritic cell. Thus one suitable assay comprises incubating a T cell with an antigen presenting cell in the presence or absence of a candidate substance and determining whether T cell proliferation is reduced or enhanced in the presence of the substance compared in the absence of the substance.

Another suitable assay involves activating a T cell with a mitogen in the presence and absence of a candidate substance and determining whether T cell proliferation is reduced or enhanced in the presence of the substance compared in the absence of the substance. Examples of suitable mitogens include monoclonal antibodies to CD3 or the TCR, phorbol 12-myrstate 13-acetate, ionomycin, concanavalin A, phytohemagglutinin, superantigens and antibodies to CD2, CD3 or the T cell receptor.

T cell activation/proliferation may be measured using a variety of techniques, for example by measuring cell number, [³H]thymidine incorporation levels of secreted cytokines such as IL-2 in the culture medium or by flow cytometric analysis of T cell surface markers indicative for activation (such as CD69, CD30, CD25 and HLA-DR).

A preferred antagonist is capable of reducing T cell proliferation by at least 50%, more preferably at least 60, 70, 80 or 90% (for example with respect to numbers of cells expressing a cell surface marker, cytokine levels in the medium and/or numbers of cells present).

Compounds for use in the present invention may be capable of inducing or preventing the NAADP+-inhibition of TCR/CD3 associated signalling. Such an effect may lead to the induction or prevention of anergy in the T cell. Determination of whether a T cell is in the anergic state can be performed using methods known in the art. For example, the T cell can be subsequently challenged with an antigen-presenting cell, and T cell activation/proliferation measured by the methods given above.

H. Therapeutic Uses

The compounds of the present invention may be used in therapy. In particular such compounds may be used to modulate T cell responses in vivo. Alternatively, T cells may be removed from a patient, treated (either with a compound of the present invention or with NAADP+ or a bioisostere thereof, or a precursor thereof, directly) and then returned to the patient (ex vivo therapy).

Compounds capable of agonising a NAADP+-mediated rise in Ca²⁺ levels, or preventing NAADP+-mediated inhibition of TCR/CD3-associated Ca²⁺ signalling in T cells may be used in methods where stimulation of T cell responses, proliferation and/or differentiation is required.

The first group of compounds may be used against any disorder which is susceptible to prevention or treatment by the induction of an adaptive immune response. In particular, these compounds may be used to treat immunodeficiency disorders mechanistically related to a defect in T cell activation.

Compounds capable of antagonising a NAADP+-mediated rise in Ca²⁺ levels, or inducing NAADP+-mediated inhibition of TCR/CD3-associated Ca²⁺ signalling in T cells may be used to treat or prevent conditions associated with an inappropriate T cell response, for example in treating autoimmune diseases, graft rejection or allergies.

Examples of disorders that may be treated by the second group of compounds include a group commonly called autoimmune diseases. The spectrum of autoimmune disorders ranges from organ specific diseases (such as thyroiditis, insulitis, multiple sclerosis, iridocyclitis, uveitis, orchitis, hepatitis, Addison's disease, myasthenia gravis) to systemic illnesses such as rheumatoid arthritis or lupus erythematosus. Other disorders include immune hyperreactivity, such as allergic reactions.

In more detail: Organ-specific autoimmune diseases include multiple sclerosis, insulin dependent diabetes mellitus, several forms of anemia (aplastic, hemolytic), autoimmune hepatitis, thyroiditis, insitis, iridocyclitis, skleritis, uveitis, orchitis, myasthenia gravis, idiopathic thrombocytopenic purpura, inflammatory bowel diseases (Crohn's disease, ulcerative colitis).

Systemic autoimmune diseases include: rheumatoid arthritis, juvenile arthritis, scleroderma and systemic sclerosis, sjogren's syndrom, undifferentiated connective tissue syndrom, antiphospholipid syndrom, different forms of vasculitis (polyarteritis nodosa, allergic granulomatosis and angiitis, Wegner's granulomatosis, Kawasaki disease, hypersensitivity vasculitis, Henoch-Schoenlein purpura, Behoet's Syndrome, Takayasu arteritis, Giant cell arteritis, Thrombangiitis obliterans), lupus erythematosus, polymyalgia rheumatica, essentiell (mixed) cryoglobulinemia, Psoriasis vulgaris and psoriatic arthritis, diffus fasciitis with or without eosinophilia, polymyositis and other idiopathic inflammatory myopathies, relapsing panniculitis, relapsing polychondritis, lymphomatoid granulomatosis, erythema nodosum, ankylosing spondylitis, Reiter's syndrom, different forms of inflammatory dermatitis.

A more extensive list of disorders is given in WO-A-98/09985. For ease of reference, part of that list is now provided: unwanted immune reactions and inflammation including arthritis, including rheumatoid arhritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery or organ, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

I. Administration

Compounds capable of affecting a NAADP+-mediated rise in Ca²⁺ levels in T cells for use in immunotherapy are typically formulated for administration to patients with a pharmaceutically acceptable carrier or diluent to produce a pharmaceutical composition. The formulation will depend upon the nature of the compound identified and the route of administration but typically they can be formulated for topical, parenteral, intramuscular, intravenous, intra-peritoneal, intranasal inhalation, lung inhalation, intradermal or intra-articular administration The compound may be used in an injectable form. It may therefore be mixed with any vehicle which is pharmaceutically acceptable for an injectable formulation, preferably for a direct injection at the site to be treated, although it may be administered systemically.

The pharmaceutically acceptable carrier or diluent may be, for example, sterile isotonic saline solutions, or other isotonic solutions such as phosphate-buffered saline. The compounds of the present invention may be admixed with any suitable binder(s), lubricant(s), suspending agent(s), coating agent(s), solubilising agent(s). It is also preferred to formulate the compound in an orally active form.

In general, a therapeutically effective daily oral or intravenous dose of the compounds of the invention, including compounds of formula (1) and their salts, is likely to range from 0.01 to 50 mg/kg body weight of the subject to be treated, preferably 0.1 to 20 mg/kg. The compounds of the formula (I) and their salts may also be administered by intravenous infusion, at a dose which is likely to range from 0.001-10 mg/kg/hr.

Tablets or capsules of the compounds may be administered singly or two or more at a time, as appropriate. It is also possible to administer the compounds in sustained release formulations.

Typically, the physician will determine the actual dosage which will be most suitable for an individual patient and it will vary with the age, weight and response of the particular patient. The above dosages are exemplary of the average case. There can, of course, be individual instances where higher or lower dosage ranges are merited, and such are within the scope of this invention.

Alternatively, the compounds of the invention can be administered by inhalation or in the form of a suppository or pessary, or they may be applied topically in the form of a lotion, solution, cream, ointment or dusting powder. An alternative means of transdermal administration is by use of a skin patch. For example, they can be incorporated into a cream consisting of an aqueous emulsion of polyethylene glycols or liquid paraffin. They can also be incorporated, at a concentration of between 1 and 10% by weight, into an ointment consisting of a white wax or white soft paraffin base together with such stabilisers and preservatives as may be required.

For some applications, preferably the compositions are administered orally in the form of tablets containing excipients such as starch or lactose, or in capsules or ovules either alone or in admixture with excipients, or in the form of elixirs, solutions or suspensions containing flavouring or colouring agents.

The compositions (as well as the compounds alone) can also be injected parenterally, for example intracavernosally, intravenously, intramuscularly or subcutaneously. In this case, the compositions will comprise a suitable carrier or diluent.

For parenteral administration, the compositions are best used in the form of a sterile aqueous solution which may contain other substances, for example enough salts or monosaccharides to make the solution isotonic with blood.

For buccal or sublingual administration the compositions may be administered in the form of tablets or lozenges which can be formulated in a conventional manner.

For oral, parenteral, buccal and sublingual administration to subjects (such as patients), the daily dosage level of the compounds of the present invention and their pharmaceutically acceptable salts and solvates may typically be from 10 to 500 mg (in single or divided doses). Thus, and by way of example, tablets or capsules may contain from 5 to 100 mg of active compound for administration singly, or two or more at a time, as appropriate. As indicated above, the physician will determine the actual dosage which will be most suitable for an individual patient and it will vary with the age, weight and response of the particular patient. It is to be noted that whilst the above-mentioned dosages are exemplary of the average case there can, of course, be individual instances where higher or lower dosage ranges are merited and such dose ranges are within the scope of this invention.

T cells treated ex vivo are typically administered to the patient by intramuscular, intraperitoneal or intravenous injection, or by direct injection into the lymph nodes of the patient, preferably by direct injection into the lymph nodes. Typically from 10⁴ to 10⁸ treated cells, preferably from 10⁵ to 10⁷ cells, more preferably about 10⁶ cells are administered to the patient.

The routes of administration and dosages described are intended only as a guide since a skilled practitioner will be able to determine readily the optimum route of administration and dosage for any particular patient depending on, for example, the age, weight and condition of the patient.

J. Solvates.

The present invention also includes the use of solvate forms of the compound of the present invention. The terms used in the claims encompass these forms.

K Pro-Drug.

As indicated, the present invention also includes the use of pro-drug forms of the compounds of the present invention. The terms used in the claims encompass these forms. Examples of prodrugs include entities that have certain protected group(s) and which may not possess pharmacological activity as such, but may, in certain instances, be administered (such as orally or parenterally) and thereafter metabolised in the body to form the compounds of the present invention which are pharmacologically active.

It will be further appreciated that certain moieties known as “pro-moieties”, for example as described in “Design of Prodrugs” by H. Bundgaard, Elsevier, 1985 (the disclosure of which is hereby incorporated by reference), may be placed on appropriate functionalities of the compounds. Such prodrugs are also included within the scope of the invention.

L. Mimetic.

In one embodiment of the present invention, the compound may be a mimetic. As used herein, the term “mimetic” relates to any chemical which includes, but is not limited to, a peptide, polypeptide, antibody or other organic chemical which has the same qualitative activity or effect as a reference agent.

M. Chemical Derivative.

In one embodiment of the present invention, the compound may be a derivative. The term “derivative” as used herein includes chemical modification of a compound. Illustrative of such chemical modifications would be replacement of hydrogen by a halo group, an alkyl group, an acyl group or an amino group.

N. Pharmaceutical Salts.

The compounds of the present invention may be administered as pharmaceutically acceptable salts. Typically, a pharmaceutically acceptable salt may be readily prepared by using a desired acid or base, as appropriate. The salt may precipitate from solution and be collected by filtration or may be recovered by evaporation of the solvent.

Pharmaceutically-acceptable salts are well known to those skilled in the art, and for example include those mentioned by Berge et al, in J. Pharm. Sci. 66, 1-19 (1977). Suitable acid addition salts are formed from acids which form non-toxic salts and include the hydrochloride, hydrobromide, hydroiodide, nitrate, sulphate, bisulphate, phosphate, hydrogenphosphate, acetate, trifluoroacetate, gluconate, lactate, salicylate, citrate, tartrate, ascorbate, succinate, maleate, fumarate, gluconate, formate, benzoate, methanesulphonate, ethanesulphonate, benzenesulphonate and p-toluenesulphonate salts.

When one or more acidic moieties are present, suitable pharmaceutically acceptable base addition salts can be formed from bases which form non-toxic salts and include the aluminium, calcium, lithium, magnesium, potassium, sodium, zinc, and pharmaceutically-active amines such as diethanolamine, salts.

The compounds of the present invention may exist in polymorphic form.

In addition, the compounds of the present invention may contain one or more asymmetric carbon atoms and therefore exists in two or more stereoisomeric forms. Where a compound contains an alkenyl or alkenylene group, cis (E) and trans (Z) isomerism may also occur. The present invention includes the individual stereoisomers of the compound and, where appropriate, the individual tautomeric forms thereof, together with mixtures thereof.

Separation of diastereoisomers or cis and trans isomers may be achieved by conventional techniques, e.g. by fractional crystallisation, chromatography or H.P.L.C. of a stereoisomeric mixture of the agent or a suitable salt or derivative thereof. An individual enantiomer of the compound may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

The present invention also includes all suitable isotopic variations of the compound or a pharmaceutically acceptable salt thereof. An isotopic variation of a compound of the present invention or a pharmaceutically acceptable salt thereof is defined as one in which at least one atom is replaced by an atom having the same atomic number but an atomic mass different from the atomic mass usually found in nature. Examples of isotopes that can be incorporated into the compound and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the compound and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the compound of the present invention and pharmaceutically acceptable salts thereof of this invention can generally be prepared by conventional procedures using appropriate isotopic variations of suitable reagents.

The present invention also includes (wherever appropriate) the use of zwitterionic forms of the compounds of the present invention.

The terms used in the claims encompass one or more of the forms just mentioned.

O. Formulation.

The component(s) of the present invention may be formulated into a pharmaceutical composition, such as by mixing with one or more of a suitable carrier, diluent or excipient, by using techniques that are known in the art.

P. Pharmaceutical Compositions.

The present invention provides a pharmaceutical composition comprising a therapeutically effective amount of one or more compounds of the present invention and a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Examples of suitable carriers include lactose, starch, glucose, methyl cellulose, magnesium stearate, mannitol, sorbitol and the like. Examples of suitable diluents include ethanol, glycerol and water.

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be administered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be administered by a number of routes.

Where the composition is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

For some embodiments, one or more compounds may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

The pharmaceutical composition may comprise one or more additional pharmaceutically active compounds.

The present invention will now be described by way of examples which are intended to be illustrative only and non-limiting.

### BRIEF DESCRIPTION OF THE FIGURES

FIG. 1(A-P)—Concentration-response curves of Ca²⁺ signals in T cells microinjected with NAADP+

FIG. 2(A,B)—Dose response curve for NAADP+ in Jurkat T cells

FIG. 3(A,B)—Concentration-response curves of Ca²⁺ signals in T cells microinjected with NAADP+ or NADP+

FIG. 4(A-J)—Concentration-response curves of Ca²⁺ signals in T cells microinjected with NAADP+ and 8-OCH3-cADPR or cADPR

FIG. 5(A-J)—Concentration-response curves of Ca²⁺ signals in T cells microinjected with NAADP+ and Ins(1,4,5)P₃ or Ins(1,4,6)PS₃

FIG. 6(A-C)—Concentration-response curves of Ca²⁺ signals in T cells microinjected with NAADP+ and then challenged with OKT3

### DETAILED DESCRIPTION OF THE FIGURES

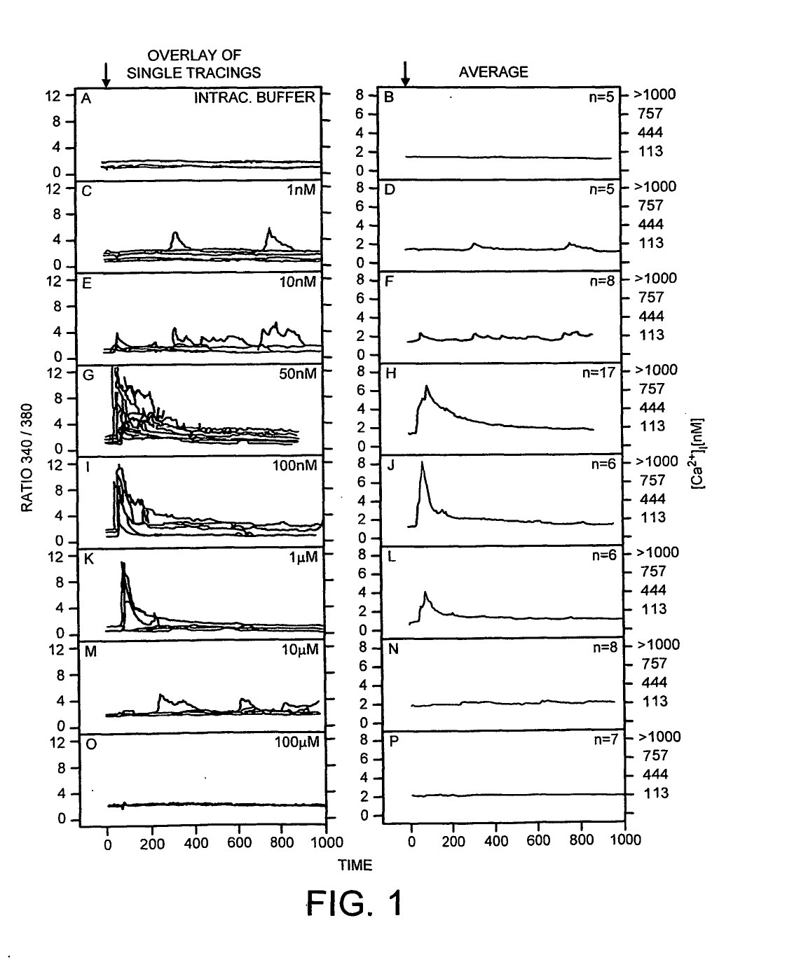

FIG. 1. Concentration-Response Curves of Ca²⁺ Signals in Single Intact T-Lymphocytes Microinjected with NAADP⁺.

Jurkat T-lymphocytes were loaded with Fura2/AM and Ca²⁺ was measured as detailed under ‘Experimental Procedures’. Cells were injected as described in Experimental Procedures’ in the presence of 1 mM extacellular Ca²⁺. Data are presented as overlays of single tracings of individual cells (left panel). The right panel shows the corresponding averages from these measurements (number of cells displayed, n=5 to 17). As a control vehicle buffer without NAADP⁺ was injected (A,B). The time point of microinjection is indicated by arrows.

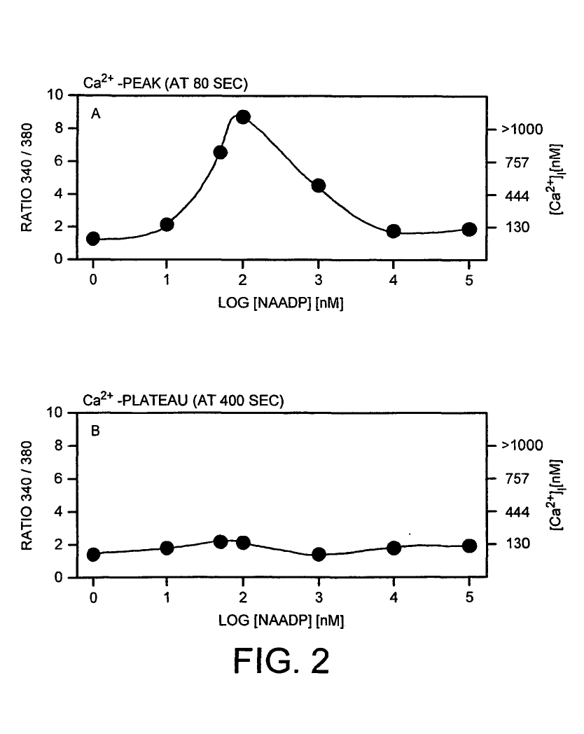

FIG. 2. Dose-Response Curve for NAADP⁺ in Jurkat T Cells.

Data from FIG. 1 are shown as mean values (n=5 to 17) from time point 80 sec (Ca²⁺ peak; A) or 400 sec (Ca²⁺ plateau; B).

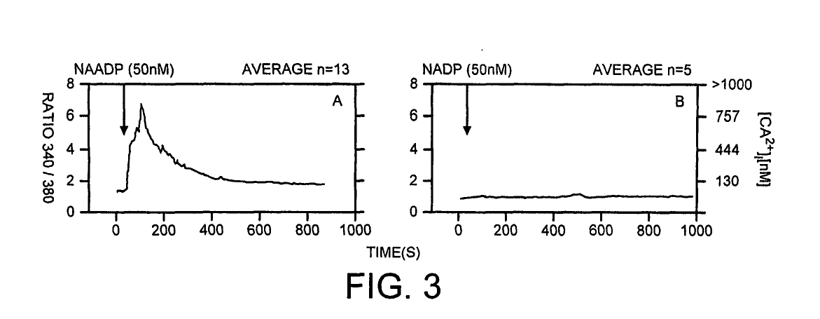

FIG. 3. NADP⁺ Does Not Mediate Ca²⁺-Signaling

Jurkat T-lymphocytes were loaded with Fura2/AM and ratiometric Ca²⁺ imaging and parallel microinjection in the presence of 1 mM extracellular Ca²⁺ was carried out as detailed under ‘Experimental Procedures’. Cells were injected with 50 nM NAADP⁺ (A) or NADP⁺ (B). Shown are the averages from 13 (A) and 5 (B) cells. The time point of microinjection is indicated by arrows.

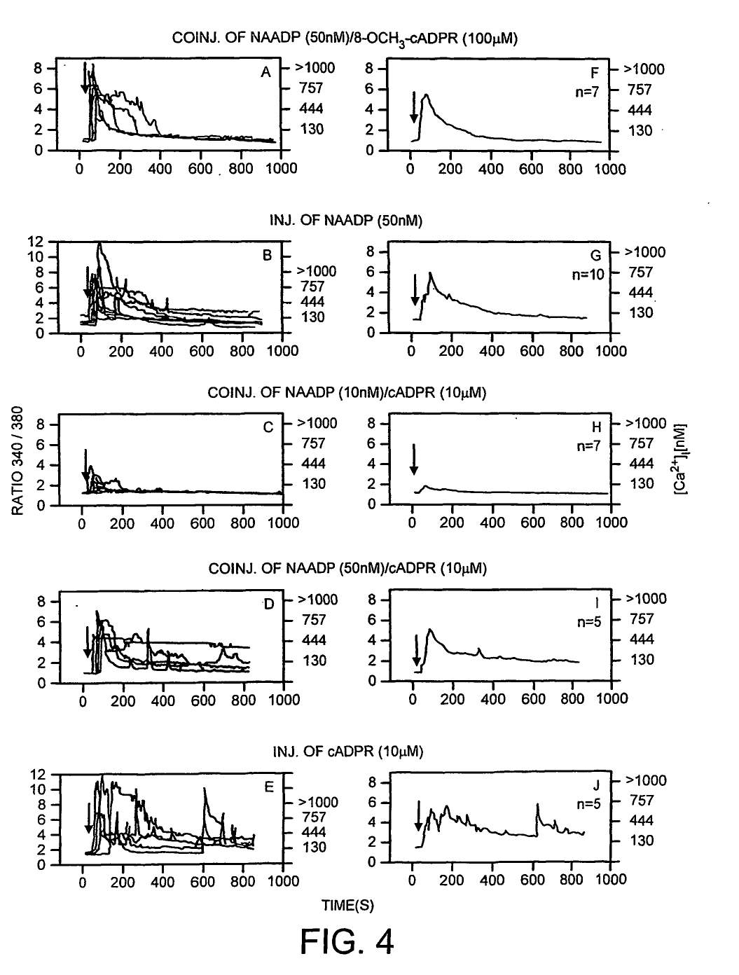

FIG. 4 Influence of cADPR and Its Antagonist 8-OCH₃-cADPR on NAADP⁺-Mediated Ca²⁺-Signaling.

Jurkat T-lymphocytes were loaded with Fura2/AM and ratiometric Ca²⁺ imaging and parallel microinjection in the presence of 1 mM extracellular Ca²⁺ was carried out as detailed under ‘Experimental Procedures’. Left panels show the overlays of single tracings of individual cells after injection (A-E), the right panels demonstrate the corresponding averages from these overlays (F-J). Shown are (n=number of experiments): A/F, coinjection of NAADP (50 nM) and 8-OCH₃-cADPR (100 μM) (n=7); B/G, injection of NAADP⁺ (50 nM, n=10); C/H, coinjection of NAADP⁺ (10 μM) and cADPR (10 μM) (n=7); D/I, coinjection of NAADP⁺ (50 nM) and cADPR (10 μM) (n=5); E/J, injection of cADPR (10 μM, n=5).

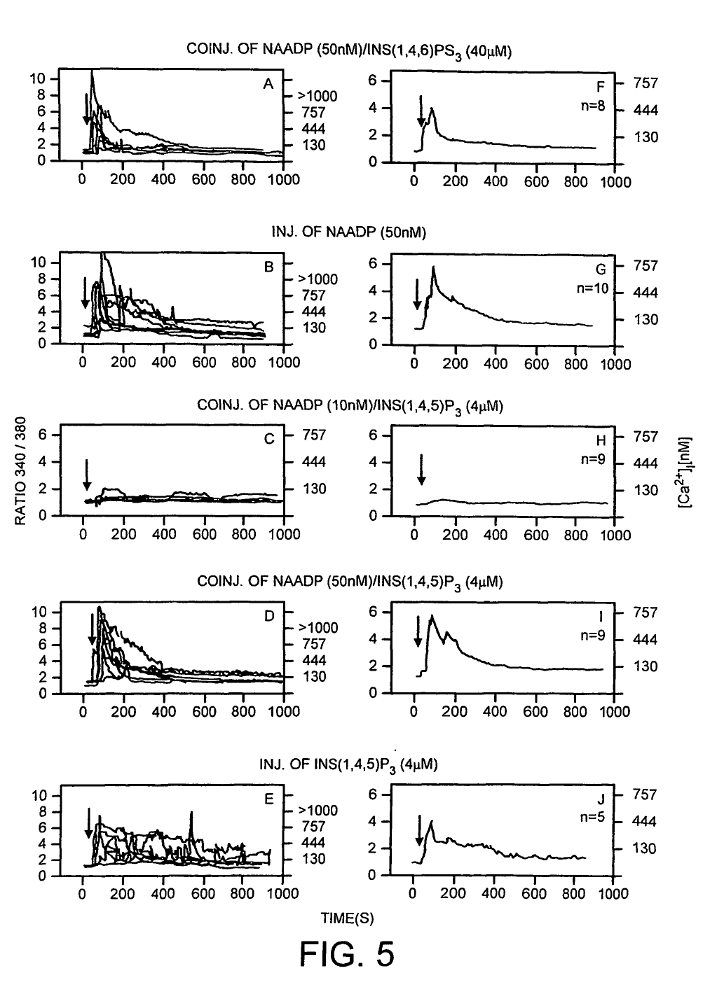

FIG. 5. Influence of Ins(1,4,5)P₃ and Its Antagonist Ins(1,4,6)PS₃ on NAADP⁺-Mediated Ca²⁺-Signaling.

Jurkat T-lymphocytes were loaded with Fura2/AM and ratiometric Ca²⁺ imaging and parallel microinjection in the presence of 1 mM extracellular Ca²⁺ was carried out as detailed under ‘Experimental Procedures’. Left panels show the overlays of single tracings of individual cells after injection (A-E), the right panels demonstrate the corresponding averages from these overlays (F-J). Shown are: A/F, coinjection of NAADP⁺ (50 nM) and Ins(1,4,6)PS₃ (40 μM) (n=7); B/G, injection of NAADP⁺ (50 nM) (n=10); C/H, coinjection of NAADP⁺ (10 μM) and Ins(1,4,5)P₃ (4 μM) (n=9); D/I, coinjection of NAADP⁺ (50 nM) and Ins(1,4,5)P₃ (4 μM) (n=3); E/J injection of Ins(1,4,5)P₃ (4 μM; n=8)

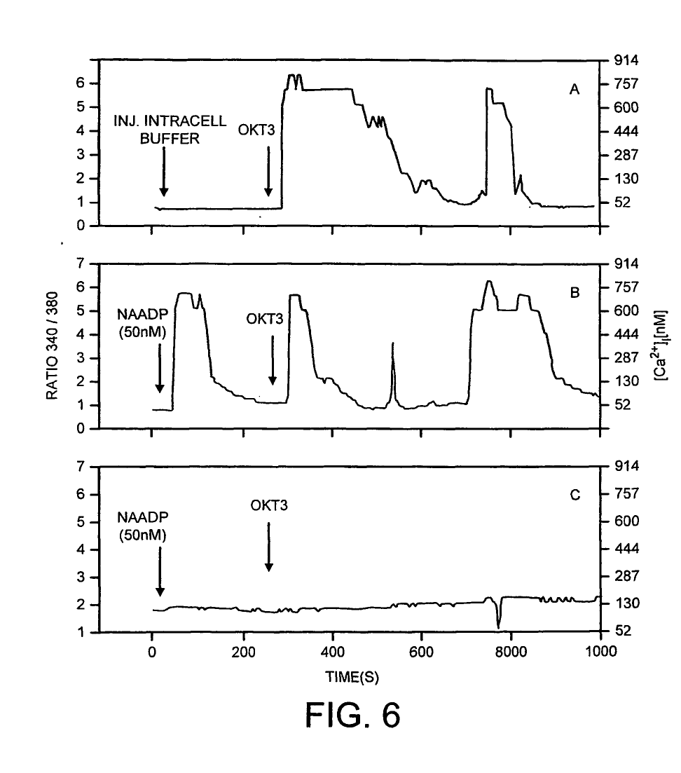

FIG. 6. Effect of NAADP⁺ on OKT3-Induced Ca²⁺ Signaling in Single Jurkat T-Lymphocytes.

Jurkat T-lymphocytes were loaded with Fura2/AM and ratiometric Ca²⁺ imaging and parallel microinjection in the presence of 1 mM extracellular Ca²⁺ was carried out as detailed under ‘Experimental Procedures’. The cells were injected with different concentrations of NAADP⁺ and then OKT3(10 μg/ml) was added. Injection of intracellular buffer (A), NAADP⁺ (50 nM [B] and 10 μM [C]), and addition of OKT3 is indicated by arrows. Data are presented as a typical tracing from one individual cell, for each condition at least 3 experiments were carried out.

### EXAMPLES

Materials and Methods

Reagents

cADPR, 8-OCH₃-cADPR, and Ins(1,4,6)PS₃ were synthesized exactly as described (Ashamu et al., 1995; Murphy et al., 2000), purified by anion-exchange chromatography on Q-Sepharose, and used as their triethylammonium salts. Purity of ligands was assessed by ¹H and ³¹P NMR spectroscopy, mass spectroscopy and, when appropriate, high performance liquid chromatography. NAADP⁺ and NADP⁺ were purchased from Sigma, Deisenhofen, Germany. The purity of NAADP⁺ was described by the manufacturer to be about 95%; this was confirmed by reverse phase HPLC using the method of da Silva et al. (1998). Fura 2/AM was obtained from Calbiochem, Bad Soden, Germany. Anti-CD3 monoclonal antibody OKT3 was purified from hybridoma supernatant on ProteinG-Sepharose.

Cell Culture

Jurkat T lymphocytes (subclone JMP) were cultured in RPMI 1640 medium containing the following additions: Glutamax I, HEPES (20 mM, pH 7.4), NCS (7.5%), penicillin (100 U/ml) and streptomycin (50 μg/ml; all obtained from Life Technologies, Eggenstein, Germany). The cells were cultured at 37° C. in a humidified atmosphere in the presence of 5% CO₂.

Ratiometric Ca²⁺-Imaging

Batches of 10⁷ Jurkat T cells were loaded with Fura2/AM as described (Guse et al., 1993). Fura2-loaded cells (10⁷ cells/5 ml) were kept at room temperature until use. Glass coverslips were coated first with bovine serum albumin (5 mg/ml), and then with poly-L-lysine (0.1 mg/ml). Small chambers consisting of a rubber O-ring were sealed on the coverslips by silicon grease. Then 90 μl of extracellular buffer (140 mM NaCl, 5 mM KCl, 1 mM MgSO₄, 1 mM CaCl₂, 20 mM Hepes, 1 mM NaH₂PO₄, 5.5 mM glucose, pH 7.4) was added, followed by addition of 10 μl cell suspension. The coverslip was mounted on the stage of an inverted microscope (Axiovert 100, Zeiss, Oberkochingen, Germany). Ratiometric Ca²⁺-imaging was performed using a PhotoMed/Photon Technology (Wedel, Germany) digital imaging system built around the Axiovert 100 microscope. Illumination at 340 and 380 nm was carried out using a chopper/optical filter system. Images were captured by an intensified CCD camera (type C2400-77; spatial resolution: 525×487 pixel; Hamamatsu, Garching, Germany) and stored as individual 340 and 380 images on harddisk. Sampling rate was usually 5 sec for a pair of images (340 and 380 nm) using 100-fold magnification. Data analysis was performed off-line using PhotoMed/Photon Technology (Wedel, Germany) “Image master” analysis software. Ratio images (340/380) were constructed pixel by pixel, and changes in the ratio over time were measured by applying “regions-of-interest” on individual cells. Finally, ratio values were converted to Ca²⁺-concentrations by external calibration.

Microinjection Experiments

Parallel Ca²⁺ imaging and microin ection experiments require a firm attachment of the Jurkat T cells without preactivation of intracellular Ca²⁺-signaling. This was achieved by the above mentioned coating procedure of the glass coverslips, as detailed earlier (Guse et al., 1997). The cells were kept in a small chamber (100 μl volume) in extracellular buffer (140 mM NaCl, 5 mM KCl, 1 mM MgSO₄, 1 mM CaCl₂, 20 mM Hepes, 1 mM NaH₂PO₄, 5.5 mM glucose, pH 7.4). Compounds to be microinjected were cleared from particles by either filtration through 0.45 μm filters, by centrifugation in an Eppendorf centrifuge at maximal speed for 10 min, or by both. Femtotips II (Eppendorf-Netheler-Hintz, Hamburg, Germany) were filled with 5 μl of reagent solution and inserted into the semi-automatic Eppendorf microinjection system (Transjector 5246, Micromanipulator 5171, Eppendorf-Netheler-Hintz, Hamburg, Germany). Injection parameters were: injection pressure: 80 hPa, compensatory pressure: 40 hPa, duration of injection: 0.5 sec, velocity of pipette: 700 μm/sec, pipette angle: 45°. Injections were performed into the upper part of the cell.

### Example 1

### NAADP+ Activates Ca²⁺ Signaling at Low Concentrations in a Dose-Dependent Manner, but High Concentrations Causes Self-Inactivation of the Ca²⁺-Release System

Microinjection of NAADP⁺ at a pipette concentration as low as 10 nM stimulated repetitive, longlasting Ca²⁺ spiking of low amplitude in intact Jurkat T cells whereas injection of intracellular buffer alone had no effect (FIG. 1A,B,E,F). Microinjection of 0.1 or 1 nM NAADP⁺ was without effect in most of the cells (FIG. 1C,D, and data not shown). At a pipette concentration of 50 nM NAADP⁺ an initial, rapidly occurring Ca²⁺-peak with a high amplitude was observed which turned into gradually lowering oscillations during the first 350 to 400 s. After this time period the calcium response changed into a low, but sustained plateau phase with very small oscillations (FIG. 1G,H). At pipette concentrations of 100 nM and 1 μM similar responses were observed (FIG. 1 I to L). However, the peak amplitude of the initial Ca²⁺-spike declined with increasing NAADP⁺ concentrations (FIG. 1 J,L), and the decay of the Ca²⁺-signal was accelerated (FIG. 1 H,J,L). At 10 μM NAADP⁺ the Ca²⁺ response appeared similar to the one at 1 nM (FIG. 1 M,N), whereas at 100 μM NAADP⁺ no signal was detectable (FIG. 1 O,P). The dose response relationship shows a bell shaped curve for the initial Ca²⁺-peak with an optimal NAADP⁺ concentration at 100 nM (FIG. 2A). However, only minor changes of the longlasting Ca²⁺-signal as measured at 400 s were observed in response to 100 nM NAADP⁺ (FIG. 2B).

These data indicate that, similar to the few other cellular systems investigated so far (Lee & Aarhus, 1995; Chini et al., 1995; Albrieux et al., 1998; Cancela et al., 1999), NAADP⁺ at low nanomolar concentrations activates Ca²⁺-signaling in T cells, whereas micromolar concentrations of NAADP⁺ rapidly cause self inactivation of the Ca²⁺-release system.

The high initial Ca²⁺-spike observed after microinjection of 50 nM NAADP⁺ was massively reduced when the extracellular Ca²⁺-concentration was decreased to a nominal Ca²⁺-free buffer indicating that Ca²⁺-entry is involved in the NAADP⁺-mediated Ca²⁺-response (data not shown).

### Example 2

### The effect of NAADP⁺ on Intracellular Ca²⁺-Signaling in T Cells is Specific

To prove the specificity of the effect of NAADP+on intracellular Ca²⁺-signaling in T cells, NADP⁺ was used in parallel microinjection experiments. NADP⁺ is a structurally very similar molecule bearing a nicotinamide group instead of the nicotinic acid group. In contrast to NAADP⁺, microinjection of NADP⁺ (50 nM) was completely without effect on Ca²⁺-signaling (FIG. 3A,B).

### Example 3

### Investigating the Relationship between the NAADP⁺ System and the Ins(1,4,5)P₃ and cADPR Systems

The Ca²⁺-release system which is targeted by NAADP⁺ has not yet been identified, but work in other cell systems indicates that neither the InsP3-R nor the RyR are involved (Lee & Aarhus, 1995; Chini et al., 1995). However, both these classical intracellular Ca²⁺-release systems have been demonstrated to be essential parts of the Ca²⁺-signaling machinery of T cells (Jayaraman et al., 1995; Guse et al., 1999). Thus, the next series of experiments were designed to investigate potential interrelations between the NAADP⁺ system on the one hand and both the Ins(1,4,5)P₃ and cADPR systems on the other hand.

The specific cADPR antagonist 8-OCH₃-cADPR (Guse et al., 1999), when co-injected with an optimal NAADP⁺ concentration, did not significantly affect NAADP⁺-mediated Ca²⁺-signaling (FIG. 4 A,F vs. B,G). However, when a self-desensitizing concentration of NAADP⁺ (10 μM) was co-injected with a stimulating concentration of CADPR (10 μM), a massive decrease of the cADPR-mediated Ca²⁺-signal was observed (FIG. 4 C,H vs. E,J). On the other hand, an optimal stimulating concentration of NAADP⁺ (50 nM) microinjected together with cADPR (10 μM) did not significantly change the Ca²⁺ signals (FIG. 4 D,I vs. E,J). These data indicate that a functional, non-desensitized NAADP⁺/Ca²⁺-release system is necessary for cADPR-mediated Ca²⁺-release.

The specific Ins(1,4,5)P₃ antagonist Ins(1,4,6)PS₃ (Guse et al., 1997; Murphy et al., 2000) was also co-injected with an optimal NAADP⁺ concentration. Surprisingly, there was a partial reduction of the initial Ca²⁺ peak, but also a faster decay of this peak as compared to injection of NAADP⁺ alone (FIG. 5A,F vs. B,G). Similar to the cADPR system, there was an almost complete inhibition of Ins(1,4,5)P₃-mediated Ca²⁺-release when a desensitizing concentration of NAADP⁺ was co-injected (FIG. 5 C,H). Co-injection of an optimal stimulating concentration of NAADP⁺ together with Ins(1,4,5)P₃ resulted in a high initial Ca²⁺ peak (FIG. 5 D,I), which was comparable to the peak observed in response to injection of NAADP⁺ alone (FIG. 5B,C), whereas much less oscillatory activity of the cells after the initial peak was observed (FIG. 5D,I) as compared to Ins(1,4,5)P₃ alone (FIG. 5 E,J). These data indicate that also the Ins(1,4,5)P₃/Ca²⁺-release system requires a functional non-desensitized NAADP⁺/Ca²⁺-release system. Moreover, a part of the Ca²⁺-signal observed in response to microinjection of NAADP⁺ alone appears to be mediated by Ins(1,4,5)P₃ (FIG. 5 A,F,B,G). This may be explained by the co-agonistic effect of Ca²⁺ released by NAADP⁺ which then acts together with basal Ins(1,4,5)P₃ at the InsP₃-R; this co-agonistic effect of Ca²⁺ at the InsP₃R has been demonstrated previously (Bezprozvanny et al., 1991).

### Example 4

### Investigating the Effect of NAADP⁺ on Ca²⁺-Signaling Mediated by an Anti-CD3 mAb

Both the Ins(1,4,5)P₃/and the cADPR/Ca²⁺-release systems have been published to be essential parts of the Ca²⁺-signaling machinery of T cells upon stimulation of the TCR/CD3 complex (Jayaraman et al., 1995; Guse et al., 1999). Since the data described above indicate that a functional NAADP⁺/Ca²⁺-release system is essential for both Ins(1,4,5)P₃- and cADPR mediated Ca²⁺-release, the present inventors investigated the effect of NAADP⁺ on Ca²⁺-signaling mediated by anti-CD3 mAb OKT3 (FIG. 6). Microinjection of 50 nM NAADP⁺ prior to stimulation of the cells by extracellular addition of OKT3 did not significantly change the OKT3-mediated Ca²⁺-signal (FIG. 6A,B). However, there was a dramatic inhibition of OKT3-mediated Ca²⁺-signaling when a desensitizing concentration of NAADP⁺ was microinjected before stimulation by OKT3 (FIG. 6C).

The main findings of this examples are (i) a dose-dependent and specific effect of NAADP⁺ in T cell Ca²⁺-signaling, (ii) the strict dependence of both Ins(1,4,5)P₃- and cADPR-mediated Ca²⁺-release upon a functional NAADP⁺/Ca²⁺-release system, and (iii) inhibition of Ca²⁺-signaling mediated by ligation of the TCR/CD3 complex by prior self inactivation of the NAADP⁺/Ca²⁺-release system.

Assuming that the NAADP⁺/Ca²⁺-release system acts as the Ca²⁺-providing trigger in T cells, the complex behavior of activation and inactivation opens a multitude of regulatory possibilities: simply by changing their endogenous NAADP⁺ concentration cells might regulate the status of the NAADP⁺/Ca²⁺-release system, e.g. by increasing NAADP⁺ the NAADP⁺/Ca²⁺-release system will become inactivated, and Ca²⁺-signaling will in turn be completely unresponsive. For T-lymphocytes, such behavior of unresponsiveness to antigenic or mitogenic stimulation is well known as anergy (Jenkins et al., 1987); however, to date it has been less clear which intracellular mechanism is responsibly involved. The present data indicate that the NAADP⁺/Ca²⁺-release system with its complex inactivation/activation properties may well be a mechanism underlying anergy in T cells.

### SUMMARY

The present invention will now be summarised by way of numbered paragraphs:

1. A method for modulating T cell activity, which comprises the step of modulating the intracellular concentration of NAADP+ or a bioisostere thereof.

2. A method according to paragraph 1, which comprises the step of stimulating a rise in intracellular Ca²⁺ levels by raising the intracellular concentration of NAADP+, or a bioisostere thereof, to an activating concentration.

3. A method according to paragraph 1, which comprises the step of inhibiting TCR/CD3-associated Ca²⁺ signalling by raising the intracellular concentration of NAADP+, or a bioisostere thereof, to an inactivating concentration.

4. A compound capable of antagonising the NAADP+-mediated rise in intracellular Ca²⁺ levels in a T cell, said rise being in response to stimulation of the T cell receptor/CD3 complex, for use in modulating T cell activity.

5. A compound capable of inducing the NAADP+-mediated inhibition of TCR/CD3-associated Ca²⁺ signalling, for use in modulating T cell activity.

6. A compound according to paragraph 5, which is capable of raising the intracellular concentration of NAADP+, or a bioisostere thereof, to an inactivating concentration.

7. A compound according to any of paragraphs 4 to 6, for use in inducing T cell anergy.

8. A compound according to any of paragraphs 4 to 6, for use in blocking T cell proliferation.

9. A compound capable of agonising the NAADP+-mediated rise in intracellular Ca²⁺ levels in a T cell, said rise being in response to stimulation of the T cell receptor/CD3 complex, for use in modulating T cell activity.

10. A compound capable of preventing the NAADP+-mediated inhibition of TCR/CD3-associated Ca²⁺ signalling, for use in modulating T cell activity.

11. A compound according to paragraphs 9 or 10, for stimulating T cell proliferation and/or differentiation.

12. A compound according to any one of paragraphs 4-11 wherein said compound is a NAADP analogue.

13. A compound according to paragraph 12 wherein the NAADP analogue has the formula (I): 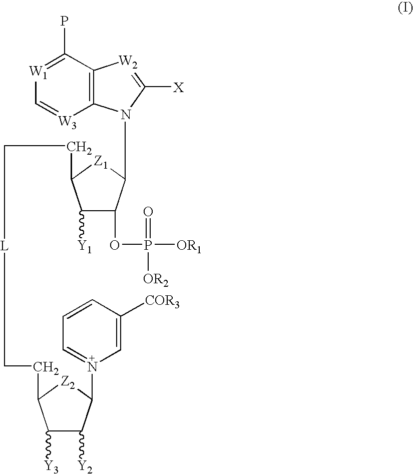
wherein P is a substituent group independently selected from NH₂, OH, SH;
each of W1, W2 or W3 is independently selected from either a CH or a heteroatom, such as N, P, S or O, preferably N;
X is a substituent group independently selected from OH, SH, NH₂, or a halo group (preferably Br);
each of R₁, R₂ or R₃ is independently selected from H or 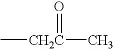
each of Y₁, Y₂ or Y₃ is independently selected from OH, H, NH₂, halo (preferably F), SH, OR₄, or 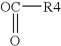
wherein R₄ is a hydrocarbyl;
each of Z₁ or Z₂ is independently selected from O, S, CH₂ or a halo derivative thereof, preferably CF₂;
and L is a linker group, suitably the linker group may have the formula (II): 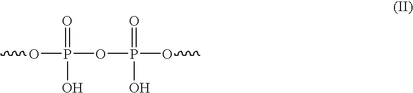
   or may be selected from one ore more the group comprising: a phosphate, a polyphosphate, a phosphorothioate, a polyethylene glycol, an alkyl, an alkylaryl, a peptide and a polyamine;
   or isomeric forms of the compound of Formula (I).

14. A compound according to paragraph 12 or paragraph 13 wherein said NAADP analogue is 8-bromo-nicotinic acid adenine dinucleotide phosphate.

15. Use of a compound as defined in any one of paragraphs 4 to 14 in the manufacture of a medicament for use in modulating the immune response of a mammal.

16. Use of a compound as defined in any one of paragraphs 4 to 8 in the manufacture of a medicament for use in treating an autoimmune disease or graft rejection.

17. Use according to paragraph 16 wherein the autoimmune disease is selected from thyroiditis, insulitis, multiple sclerosis, iridocyclitis, uveitis, orchitis, hepatitis, Addison's disease, myasthenia gravis, rhematoid arthritis and lupus erythematosus.

18. Use of a compound as defined in any one of paragraphs 4 to 17 in the manufacture of a medicament for use in treating or preventing an immune disorder in a human or animal.

19. A method of treating or preventing a disease in a human or animal patient which method comprises administering to the patient an effective amount of a compound as defined in any one of paragraphs 4 to 14.

20. A method for identifying a substance capable of antagonising the NAADP+-mediated rise in intracellular Ca²⁺ levels in a T cell, which method comprises:
(i) contacting a T cell, which has been stimulated via its T cell receptor, with a candidate substance under conditions that would permit a rise in intracellular Ca²⁺ levels in the absence of the substance; and
(ii) determining whether the substance inhibits a rise in intracellular Ca²⁺ levels.

21. A method for identifying a substance capable of inducing the NAADP+-mediated inhibition of TCR/CD3-associated Ca²⁺ signalling, which method comprises:
(i) contacting a T cell with a candidate substance;
(ii) stimulating the T cells via TCR/CD3; and
(ii) determining whether the substance inhibits TCR/CD3-associated Ca²⁺ signalling.

22. A method for identifying a substance capable of agonising the NAADP+-mediated rise in intracellular Ca²⁺ levels in a T cell, which method comprises:
(i) contacting a T cell with a candidate substance; and
(ii) determining whether the substance elicits or enhances a rise in intracellular NAADP+ and/or Ca²⁺ levels.

23. A substance identified by the method of paragraph 20, 21 or 22.

24. A process comprising the steps of:
(a) performing the method according to paragraph 20, 21 or 22;
(b) preparing a quantity of one or more substances identified by the method.

25. A compound capable of modulating the intracellular concentration and/or the binding affinity of NAADP+ wherein the compound is a NAADP analogue.

26. A compound capable of modulating the intracellular concentration and/or the binding affinity of NAADP+ wherein the compound has the formula (I): 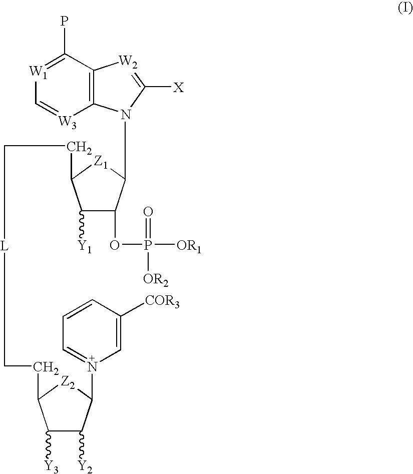
wherein P is a substituent group independently selected from NH₂, OH, SH;
each of W1, W2 or W3 is independently selected from either a CH or a heteroatom, such as N, P, S or O, preferably N;
X is a substituent group independently selected from OH, SH, NH₂, or a halo group (preferably Br);
each of R₁, R₂ or R₃ is independently selected from H or 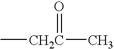
each of Y₁, Y₂ or Y₃ is independently selected from OH, H, NH₂, halo (preferably F), SH, OR₄, or 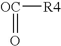
wherein R₄ is a hydrocarbyl;
each of Z₁ or Z₂ is independently selected from O, S, CH₂ or a halo derivative thereof, preferably CF₂;
and L is a linker group, suitably the linker group may have the formula (II): 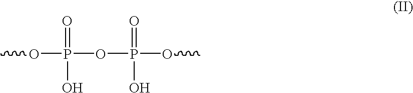
   or may be selected from one ore more the group comprising: a phosphate, a polyphosphate, a phosphorothioate, a polyethylene glycol, an alkyl, an alkylaryl, a peptide and a polyamine;
   or isomeric forms of the compound of Formula (I).

27. A compound capable of modulating the intracellular concentration and/or the binding affinity of NAADP+ wherein the compound is 8-bromo-nicotinic acid adenine dinucleotide phosphate.

28. Use of a NAADP analogue in the manufacture of a medicament for use in modulating the immune response of a mammal.

29. Use of a compound having formula (I): 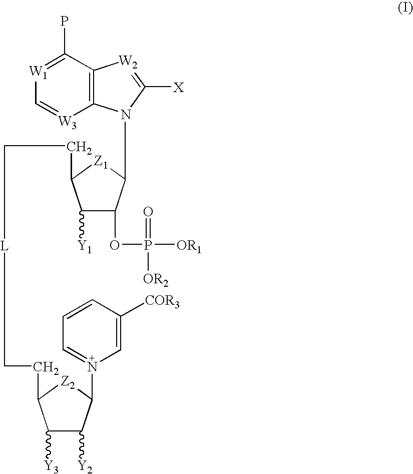
wherein P is a substituent group independently selected from NH₂, OH, SH;
each of W1, W2 or W3 is independently selected from either a CH or a heteroatom, such as N, P, S or O, preferably N;
X is a substituent group independently selected from OH, SH, NH₂, or a halo group (preferably Br);
each of R₁, R₂ or R₃ is independently selected from H or 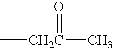
each of Y₁, Y₂ or Y₃ is independently selected from OH, H, NH₂, halo (preferably F), SH, OR₄, or 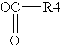
wherein R₄ is a hydrocarbyl;
each of Z₁ or Z₂ is independently selected from O, S, CH₂ or a halo derivative thereof, preferably CF₂;
and L is a linker group, suitably the linker group may have the formula (II): 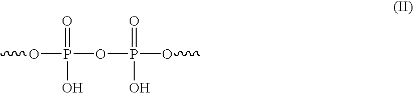
   or may be selected from one ore more the group comprising: a phosphate, a polyphosphate, a phosphorothioate, a polyethylene glycol, an alkyl, an alkylaryl, a peptide and a polyamine;
   or isomeric forms of the compound of Formula (I)
   in the manufacture of a medicament for use in modulating the immune response of a mammal.

30. Use of 8-bromo-nicotinic acid adenine dinucleotide phosphate in the manufacture of a medicament for use in modulating the immune response of a mammal.

31. Use of a NAADP analogue in the manufacture of a medicament for use in treating an autoimmune disease or graft rejection.

32. Use of a compound having formula (I): 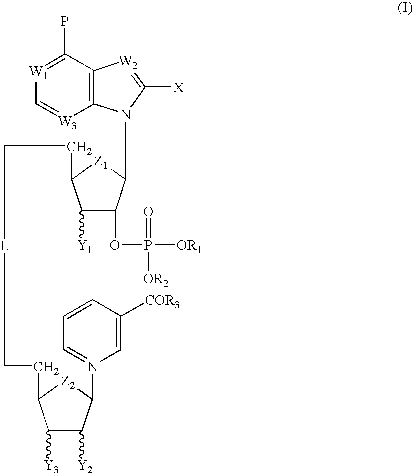
wherein P is a substituent group independently selected from NH₂, OH, SH;
each of W1, W2 or W3 is independently selected from either a CH or a heteroatom, such as N, P, S or O, preferably N;
X is a substituent group independently selected from OH, SH, NH₂, or a halo group (preferably Br);
each of R₁, R₂ or R₃ is independently selected from H or 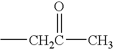
each of Y₁, Y₂ or Y₃ is independently selected from OH, H, NH₂, halo (preferably F), SH, OR₄, or 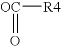
wherein R₄ is a hydrocarbyl;
each of Z₁ or Z₂ is independently selected from O, S, CH₂ or a halo derivative thereof, preferably CF₂;
and L is a linker group, suitably the linker group may have the formula (II): 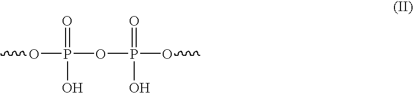
   or may be selected from one ore more the group comprising: a phosphate, a polyphosphate, a phosphorothioate, a polyethylene glycol, an alky, an alkylaryl, a peptide and a polyamine;
   or isomeric forms of the compound of Formula (I)
   in the manufacture of a medicament for use in treating an autoimmune disease or graft rejection.

33. Use of 8-bromo-nicotinic acid adenine dinucleotide phosphate in the manufacture of a medicament for use in treating an autoimmune disease or graft rejection.

34. A method of treating or preventing a disease in a human or animal patient which method comprises administering to the patient an effective amount of a NAADP analogue.

35. A method of treating or preventing a disease in a human or animal patient which method comprises administering to the patient an effective amount of a compound having formula (I): 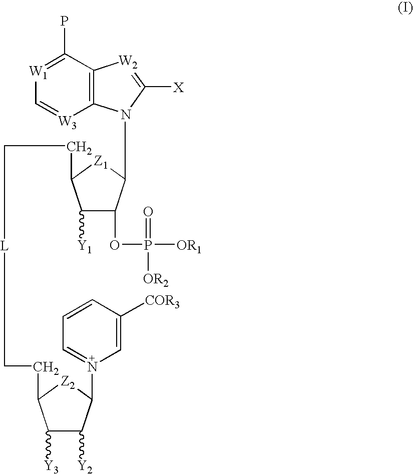
wherein P is a substituent group independently selected from NH₂, OH, SH;
each of W1, W2 or W3 is independently selected from either a CH or a heteroatom, such as N, P, S or O, preferably N;
X is a substituent group independently selected from OH, SH, NH₂, or a halo group (preferably Br);
each of R₁, R₂ or R₃ is independently selected from H or 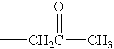
each of Y₁, Y₂ or Y₃ is independently selected from OH, H, NH₂, halo (preferably F), SH, OR₄, or 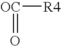
wherein R₄ is a hydrocarbyl;
each of Z₁ or Z₂ is independently selected from O, S, CH₂ or a halo derivative thereof, preferably CF₂;
and L is a linker group, suitably the linker group may have the formula (II): 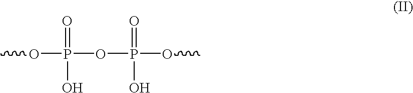
   or may be selected from one ore more the group comprising: a phosphate, a polyphosphate, a phosphorothioate, a polyethylene glycol, an alky, an alkylaryl, a peptide and a polyamine;
   or isomeric forms of the compound of Formula (I).

36. A method of treating or preventing a disease in a human or animal patient which method comprises administering to the patient an effective amount of 8-bromo-nicotinic acid adenine dinucleotide phosphate.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

### REFERENCES

Albrieux, M., H. C. Lee, and M. Villaz. 1998. J. Biol. Chem. 273:14566-14574.
Bezprozvanny, I., J. Watras, and B. E. Ehrlich. 1991 Nature 351:751-754.
Cancela, J. M., G. C. Churchill, and A. Galione. 1999. Nature (Lond) 398:74-76.
Chini, E. N., K. W. Beers, and T. P. Dousa 1995. J. Biol. Chem. 270:3216-3223.
Clapper, D. L., T. F. Walseth, P. J. Dargie, and H. C. Lee. 1987. J. Biol. Chem. 262:9561-9568.
Guse, A. H., Berg, I., da Silva, C. P., Potter, B. V. L., & Mayr, G. W., J. Biol. Chem. 272, 8546-8550 (1997).
Guse, A. H., Ca²⁺ signalling in T-lymphocytes, Crit. Rev. Immunol. 18, 419-448 (1998).
Guse, A. H., et al. Nature (Lond.) 398:70-73 (1999).
Jayaraman, T., Ondriasove, E., Ondrias, K., Harnick, D. J., & Marks, A. R, Proc. Natl. Acad. Sci. USA 92, 6007-6011(1995).
Jenkins, M., D. M. Pardoll, J. Mizuguchi, T. M. Chused, and R. H. Schwartz. 1987. Proc. Natl. Acad. Sci. USA 84:5409-5413.
Lee, H. C., and R. Aarhus. 1995. J. Biol. Chem. 270:2152-2157.
Murphy, C. T., A. M. Riley. S. J. Mills, C. J. Lindley, B. V. L. Potter, and J. Westwick. 2000. Mol. Pharmacol. 57:595-601
Rahavendran, S. V., and H. T. Karnes. 1993. Pharm. Res. 10:328-34.

1. A method for modulating T cell activity, which comprises the step of modulating the intracellular concentration of NAADP+ or a bioisostere thereof.

2. A method according to claim 1, which comprises the step of stimulating a rise in intracellular Ca²⁺ levels by raising the intracellular concentration of NAADP+, or a bioisostere thereof, to an activating concentration.

3. A method according to claim 1, which comprises the step of inhibiting TCR/CD3-associated Ca²⁺ signalling by raising the intracellular concentration of NAADP+, or a bioisostere thereof, to an inactivating concentration.

4. A compound capable of antagonising the NAADP+-mediated rise in intracellular Ca²⁺ levels in a T cell, said rise being in response to stimulation of the T cell receptor/CD3 complex, for use in modulating T cell activity.

5. A compound capable of inducing the NAADP+-mediated inhibition of TCR/CD3-associated Ca²⁺ signalling, for use in modulating T cell activity

6. A compound according to claim 5, which is capable of raising the intracellular concentration of NAADP+, or a bioisostere thereof, to an inactivating concentration

7. A compound according to any of claims 4 to 6, for use in inducing T cell anergy.

8. A compound according to any of claims 4 to 6, for use in blocking T cell proliferation.

9. A compound capable of agonising the NAADP+-mediated rise in intracellular Ca²⁺ levels in a T cell, said rise being in response to stimulation of the T cell receptor/CD3 complex, for use in modulating T cell activity.

10. A compound capable of preventing the NAADP+-mediated inhibition of TCR/CD3-associated Ca²⁺ signalling, for use in modulating T cell activity

11. A compound according to claim 9 or 10, for stimulating T cell proliferation and/or differentiation.

12. A compound according to any one of claims 4-11 wherein said compound is a NAADP analogue.

13. A compound according to claim 12 wherein the NAADP analogue has the formula (I): 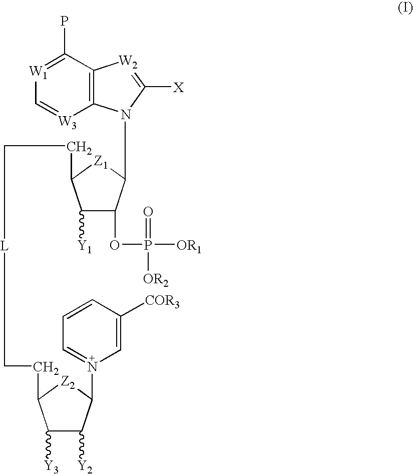
wherein P is a substituent group independently selected from NH₂, OH, SH;
each of W1, W2 or W3 is independently selected from either a CH or a heteroatom, such as N, P, S or O, preferably N;
X is a substituent group independently selected from OH, SH, NH₂, or a halo group (preferably Br);
each of R₁, R₂ or R₃ is independently selected from H or 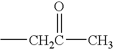
each of Y₁, Y₂ or Y₃ is independently selected from OH, H, NH₂, halo (preferably F), 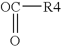
wherein R₄ is a hydrocarbyl;
each of Z₁ or Z₂ is independently selected from O, S, CH₂ or a halo derivative thereof, preferably CF₂;
and L is a linker group, suitably the linker group may have the formula (II): 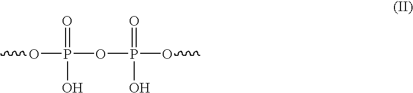
or may be selected from one ore more the group comprising: a phosphate, a polyphosphate, a phosphorothioate, a polyethylene glycol, an alkyl, an alkylaryl, a peptide and a polyamine;
or isomeric forms of the compound of Formula (I).

14. A compound according to claim 12 or claim 13 wherein said NAADP analogue is 8-bromo-nicotinic acid adenine dinucleotide phosphate.

15. Use of a compound as defined in any one of claims 4 to 14 in the manufacture of a medicament for use in modulating the immune response of a mammal.

16. Use of a compound as defined in any one of claims 4 to 8 in the manufacture of a medicament for use in treating an autoimmune disease or graft rejection.

17. Use according to claim 16 wherein the autoimmune disease is selected from thyroiditis, insulitis, multiple sclerosis, iridocyclitis, uveitis, orchitis, hepatitis, Addison's disease, myasthenia gravis, rhematoid arthritis and lupus erythematosus.

18. Use of a compound as defined in any one of claims 4 to 17 in the manufacture of a medicament for use in treating or preventing an immune disorder in a human or animal.

19. A method of treating or preventing a disease in a human or animal patient which method comprises administering to the patient an effective amount of a compound as defined in any one of claims 4 to 14.

20. A method for identifying a substance capable of antagonising the NAADP+-mediated rise in intracellular Ca²⁺ levels in a T cell, which method comprises:
(i) contacting a T cell, which has been stimulated via its T cell receptor, with a candidate substance under conditions that would permit a rise in intracellular Ca²⁺ levels in the absence of the substance; and
(ii) determining whether the substance inhibits a rise in intracellular Ca²⁺ levels.

21. A method for identifying a substance capable of inducing the NAADP+-mediated inhibition of TCR/CD3-associated Ca²⁺ signalling, which method comprises:
(i) contacting a T cell with a candidate substance;
(ii) stimulating the T cells via TCR/CD3; and
(ii) determining whether the substance inhibits TCR/CD3-associated Ca²⁺ signalling.

22. A method for identifying a substance capable of agonising the NAADP+-mediated rise in intracellular Ca²⁺ levels in a T cell, which method comprises:
(i) contacting a T cell with a candidate substance; and
(ii) determining whether the substance elicits or enhances a rise in intracellular NAADP+ and/or Ca²⁺ levels.

23. A substance identified by the method of claim 20, 21 or 22.

24. A process comprising the steps of:
(a) performing the method according to claim 20, 21 or 22;
(b) preparing a quantity of one or more substances identified by the method.

25. A compound capable of modulating the intracellular concentration and/or the binding affinity of NAADP+ wherein the compound is a NAADP analogue.

26. A compound capable of modulating the intracellular concentration and/or the binding affinity of NAADP+ wherein the compound has the formula (I): 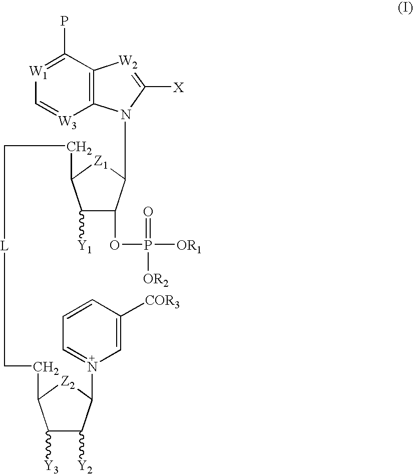
wherein P is a substituent group independently selected from NH₂, OH, SH;
each of W1, W2 or W3 is independently selected from either a CH or a heteroatom, such as N, P, S or O, preferably N;
X is a substituent group independently selected from OH, SH, NH₂, or a halo group (preferably Br);
each of R₁, R₂ or R₃ is independently selected from H or 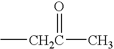
each of Y₁, Y₂ or Y₃ is independently selected from OH, H, NH₂, halo (preferably F), SH, OR₄, or 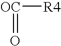
wherein R₄ is a hydrocarbyl;
each of Z₁ or Z₂ is independently selected from O, S, CH₂ or a halo derivative thereof, preferably CF₂;
and L is a linker group, suitably the linker group may have the formula (II): 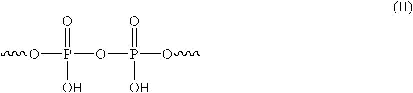
or may be selected from one ore more the group comprising: a phosphate, a polyphosphate, a phosphorothioate, a polyethylene glycol, an alkyl, an alkylaryl, a peptide and a polyamine;
or isomeric forms of the compound of Formula (I).

27. A compound capable of modulating the intracellular concentration and/or the binding affinity of NAADP+ wherein the compound is 8-bromo-nicotinic acid adenine dinucleotide phosphate.

28. Use of a NAADP analogue in the manufacture of a medicament for use in modulating the immune response of a mammal.

29. Use of a compound having formula (I): 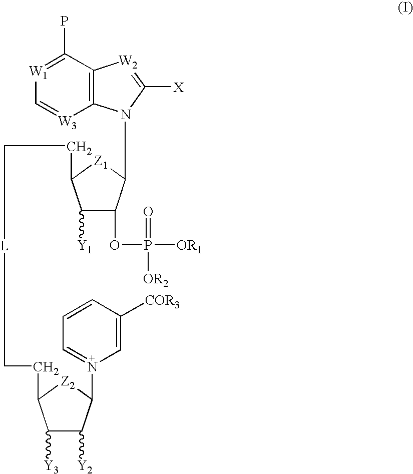
wherein P is a substituent group independently selected from NH₂, OH, SH;
each of W1, W2 or W3 is independently selected from either a CH or a heteroatom, such as N, P, S or O, preferably N;
X is a substituent group independently selected from OH, SH, NH₂, or a halo group (preferably Br);
each of R₁, R₂ or R₃ is independently selected from H or 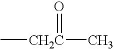
each of Y₁, Y₂ or Y₃ is independently selected from OH, H, NH₂, halo (preferably F), SH, OR₄, or 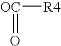
wherein R₄ is a hydrocarbyl;
each of Z₁ or Z₂ is independently selected from O, S, CH₂ or a halo derivative thereof, preferably CF₂;
and L is a linker group, suitably the linker group may have the formula (II): 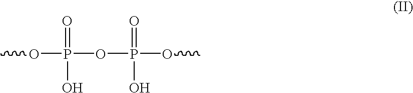
or may be selected from one ore more the group comprising: a phosphate, a polyphosphate, a phosphorothioate, a polyethylene glycol, an alkyl, an alkylaryl, a peptide and a polyamine;
or isomeric forms of the compound of Formula (I)
in the manufacture of a medicament for use in modulating the immune response of a mammal.

30. Use of 8-bromo-nicotinic acid adenine dinucleotide phosphate in the manufacture of a medicament for use in modulating the immune response of a mammal.

31. Use of a NAADP analogue in the manufacture of a medicament for use in treating an autoimmune disease or graft rejection.

32. Use of a compound having formula (I): 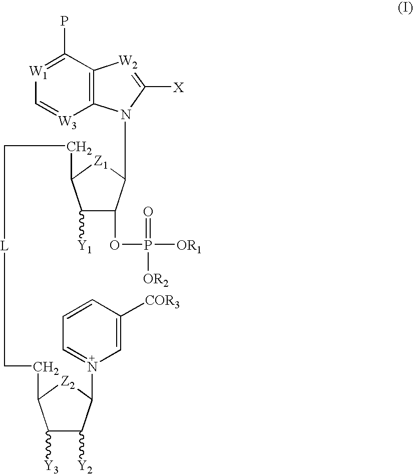
wherein P is a substituent group independently selected from NH₂, OH, SH;
each of W1, W2 or W3 is independently selected from either a CH or a heteroatom, such as N, P, S or O, preferably N;
X is a substituent group independently selected from OH, SH, NH₂, or a halo group (preferably Br);
each of R₁, R₂ or R₃ is independently selected from H or 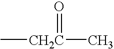
each of Y₁, Y₂ or Y₃ is independently selected from OH, H, NH₂, halo (preferably F), SH, OR₄, or 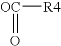
wherein R₄ is a hydrocarbyl;
each of Z₁ or Z₂ is independently selected from O, S, CH₂ or a halo derivative thereof, preferably CF₂;
and L is a linker group, suitably the linker group may have the formula (II): 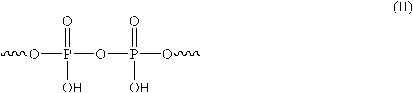
or may be selected from one ore more the group comprising: a phosphate, a polyphosphate, a phosphorothioate, a polyethylene glycol, an alkyl, an alkylaryl, a peptide and a polyamine;
or isomeric forms of the compound of Formula (I)
in the manufacture of a medicament for use in treating an autoimmune disease or graft rejection.

33. Use of 8-bromo-nicotinic acid adenine dinucleotide phosphate in the manufacture of a medicament for use in treating an autoimmune disease or graft rejection.

34. A method of treating or preventing a disease in a human or animal patient which method comprises administering to the patient an effective amount of a NAADP analogue.

35. A method of treating or preventing a disease in a human or animal patient which method comprises administering to the patient an effective amount of a compound having formula (I): 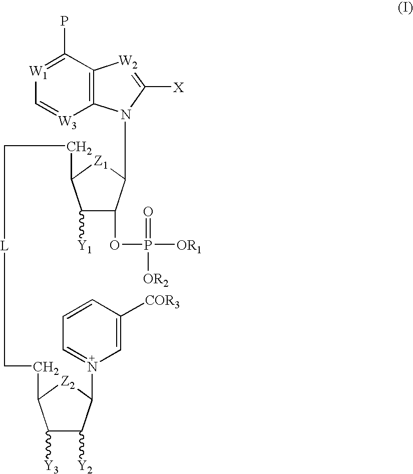
wherein P is a substituent group independently selected from NH₂, OH, SH;
each of W1, W2 or W3 is independently selected from either a CH or a heteroatom, such as N, P, S or O, preferably N;
X is a substituent group independently selected from OH, SH, NH₂, or a halo group (preferably Br);
each of R₁, R₂ or R₃ is independently selected from H or 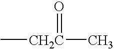
each of Y₁, Y₂ or Y₃ is independently selected from OH, H, NH₂, halo (preferably F), SH, OR₄, or 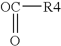
wherein R₄ is a hydrocarbyl;
each of Z₁ or Z₂ is independently selected from O, S, CH₂ or a halo derivative thereof, preferably CF₂;
and L is a linker group, suitably the linker group may have the formula (II): 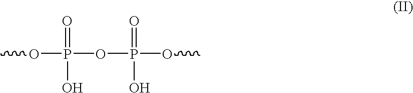
or may be selected from one ore more the group comprising: a phosphate, a polyphosphate, a phosphorothioate, a polyethylene glycol, an alkyl, an alkylaryl, a peptide and a polyamine;
or isomeric forms of the compound of Formula (I).

36. A method of treating or preventing a disease in a human or animal patient which method comprises administering to the patient an effective amount of 8-bromo-nicotinic acid adenine dinucleotide phosphate.
